(19) United States
(12) Patent Application Publication
(10) Pub. No.: US 2023/0326589 A1
(43) Pub. Date: Oct. 12, 2023

(54) SIGNAL PROCESSING FOR CARE PROVISION

(71) Applicant: LogicMark, Inc., Louisville, KY (US)

(72) Inventors: Rafael SAAVEDRA, Louisville, KY (US); Chia-Lin SIMMONS, Louisville, KY (US); Peter WILLIAMS, Louisville, KY (US)

(73) Assignee: LogicMark, Inc., Louisville, KY (US)

(21) Appl. No.: 18/129,713

(22) Filed: Mar. 31, 2023

Related U.S. Application Data
(60) Provisional application No. 63/328,083, filed on Apr. 6, 2022.

(51) Int. Cl. G16H 40/40; G16H 40/67

(52) U.S. Cl. CPC G16H 40/40; G16H 40/67

(57) ABSTRACT

A system, apparatus, and method to monitor at least one person in at least one environment. A care processing system receives data from at least one sensor in an environment for a monitoring a person under care. The data received is matched to a pattern for such data configured by the signal processing system. Upon the matching of such data, at least a first sensor creates an event that in whole or in part matches such pattern. The signal processing configures a first sensor to collect data created by such event, or instructs a second and subsequent set of sensors to collect data about the event, such that signal processing may determine the accuracy of such data for matching of the pattern. The situation is represented for a monitored person in an environment for the purpose of providing care to that person.

### PRIORITY CLAIM

This application claims the benefit of U.S. Provisional Application No. 63/328,083, entitled, “Signal Processing for Care Provision,” which was filed on Apr. 6, 2022.

### BACKGROUND

### Field of the Disclosure

Aspects of the disclosure relate in general to a system to monitor a person under care.

### Description of the Related Art

In traditional infrastructure technology environments, Personal Emergency Response Systems (PERS), also known as Medical Emergency Response Systems, allow persons to call for help in an emergency by pushing a button.

One example system is a two-way voice communication pendant that allows a person to call for assistance anywhere around their home. Personal emergency response devices make aging in place and independent living a possibility for persons under care. The personal emergency response device allows a person to remain connected with loved ones and emergency services through an existing landline telephone.

### SUMMARY

A system to monitor a person under care by a stakeholder, comprising a plurality of environmental sensors and a care processing system. The plurality of environmental sensors is configured to monitor the person under care, and to provide a detected data set representing behaviors of the person under care in an environment. Each of the behaviors is represented by a multi-dimensional feature set forming part of a health care profile for the person under care. The care processing system comprises a transceiver, a non-transitory computer-readable storage medium, and at least one hardware processing unit. The transceiver is configured to receive the detected data set. The non-transitory computer-readable storage medium is configured to store a quiescent data set. The quiescent data set represents previous quiescent behaviors of the person under care in the environment. The at least one hardware processing unit determines a wellness or care event for the person under care by comparing the detected data set and the quiescent data set. When the wellness or care event has occurred, the care processing system is configured to change a state of the plurality of environmental sensors or notify the stakeholder.

In an alternate embodiment, a system deploys a pattern representing a health state of a person under care by a stakeholder. The system comprises a plurality of environmental sensors and a care processing system. The plurality of environmental sensors is configured to monitor the person under care, and to provide a detected data set representing behaviors of the person under care in an environment. Each of the behaviors is represented by a multi-dimensional feature set forming part of a health care profile for the person under care. The care processing system comprises a transceiver, and at least one hardware processing unit. The transceiver is configured to receive the detected data set. The at least one hardware processing unit determines a variation in the detected data set indicating a transition state between a first pattern and a second pattern within the health state representing a wellness and care state of the person under care. The care processing system is configured to change a sensor configuration of the plurality of environmental sensors to adjust for the transition state.

In yet another alternate embodiment, a system monitors a person under care by a stakeholder. The system comprises a plurality of environmental sensors and a care processing system. The plurality of environmental sensors is configured to monitor the person under care, and to provide a detected data set representing a care state of the person under care in an environment. The care processing system comprises a transceiver, and at least one hardware processing unit. The transceiver is configured to receive the detected data set. The at least one hardware processing unit identifies and determines a care signal that represents the care state of the person under care. The care signal comprises a multi-dimensional feature set. The care processing system is configured to respond to the care signal involving the stakeholder.

### BRIEF DESCRIPTION OF THE DRAWINGS

These and other aspects will now be described in detail with reference to the following drawings.

FIG. 1 illustrates an example set of modules that in combination provide, at least in part, the systems for the monitoring of a person under care described herein.

FIG. 2 illustrates an HCP representing the care journey of a PUM from their initial care state, which represents the initial care condition for monitoring, through a series of care states that lead to a palliative hospice care condition and ultimately a terminal care condition.

FIG. 3 illustrates an HCP, where the PUM makes a recovery to at least the initial condition that caused them to be placed under monitoring.

FIG. 4 illustrates a set of modules for monitoring a PUM (105) in an environment.

FIG. 5 illustrates the care processing systems integrations with a set of response systems.

FIG. 6 illustrates monitoring focus modules.

FIG. 7 illustrates a transition state between two operating patterns.

FIG. 8 illustrates variation in a behavior pattern.

FIG. 9 illustrates the use of predictive and matching systems.

FIG. 10 illustrates one or more digital twins.

FIG. 11 illustrates a PERS device being worn by a PUM in an environment

FIG. 12 Illustrates a PUM in an environment that includes sets of sensors.

FIG. 13 illustrates a PUM (105) in an environment where sensors generate data sets.

FIG. 14 illustrates an embodiment of a care hub.

FIG. 15 Illustrates a PUM (105) in an environment.

FIG. 16 illustrates one or more digital twins.

### DETAILED DESCRIPTION

Aspects include a care processing system receiving data from at least one sensor in an environment for a monitoring a person under care. The data received is matched to a pattern for such data configured by the care processing system. Upon the matching of such data, at least a first sensor creates an event that in whole or in part matches such pattern. The care processing care processing configures a first sensor to collect data created by such event, or instructs a second and subsequent set of sensors to collect data about the event, such that Care processing may determine the accuracy of such data for matching of the pattern. The situation is represented for a monitored person in an environment for the purpose of providing care to that person.

Care processing requires two fundamental elements: a recognizable care signal that can be separated from the background state in which the care signal is present.

When to one or more person is predominantly domiciled in an environment, for example, an enclosed space such as a room, apartment, house and the like, this environment can be considered as providing the background in which signals representing events that may impact the care and wellness of that person, known as the person under monitoring (PUM), may be detected. As entropy is always increasing, no environment can be considered to be at a state of rest. Rather, the environment will include a certain background set of characteristics, which over time create a backdrop against which sensors can measure one or more variations in those characteristics. These variations may represent changes in the state of the environment and can be identified and/or classified as events. For example, temperature, pressure, light, radio frequency (RF) and other electromagnetic waves, humidity and other characteristics may all vary, and such variations can be represented as events.

In environments where one or more sensors are deployed, each may have a limitation as to the sensitivity of such a sensor to detect changes in the measured characteristics that the sensor is capable of measuring. In this way an individual sensor may be able to detect, for example, movement which is above the threshold of sensitivity of the device. In this manner each device has detection threshold characteristics, usually defined by the specifications of the sensor, and a field of perception or capture defining the ability of the range of the sensors sensing capabilities. Further each sensor has minimum operating characteristics, in that with no detection of variance of the environment in which it is sensing this can be represented as the quiescent state of the sensor.

An aggregation of sensors may individually be able to measure the characteristics that each is capable of, however there is no combined background that can be created other than the aggregation of the sensor data with no recognizable events for each sensor.

To effectively monitor an environment requires that each of the sensors be aligned to a common model representing the environment with no activity, such that a baseline for the state of the environment is created. This involves the collection by the sensors in an environment of data that is captured over a period of time such that any measurements include variations caused by time of day and/or other environmental factors. This data set can be augmented by external to the environment data sets, such as those of weather monitoring systems and the like.

The system may include a set of baseline measurements that are typical for various environments, for example, these may be based in part on patterns, predictions, calculations and, where available, measurements of the actual or similar environments in one or more dimensions. For example, if an environment is carpeted, the acoustic profile will differ from one with a hard surface floor.

One simple approach can involve the installation of a device carrying multiple sensors that can measure the environment, such as temperature, humidity, pressure, time of day, ambient sound level and the like. This baseline data can then be combined with environment specifications to create a model of the environment in which the behaviors of a PUM may be monitored. There are only a finite number of environment spaces that a PUM will inhabit in a domestic and/or care situation. These can be created as digital twins in, for example, modelling environments such as Unreal Engine, Unity, Autodesk or other 3D modelling systems.

Based on the configuration of those environments, for example whether they incorporate climate control, ratio of soft to hard surfaces, purpose (kitchen/bathroom etc.) and any other characteristics, a baseline for Care processing can be created.

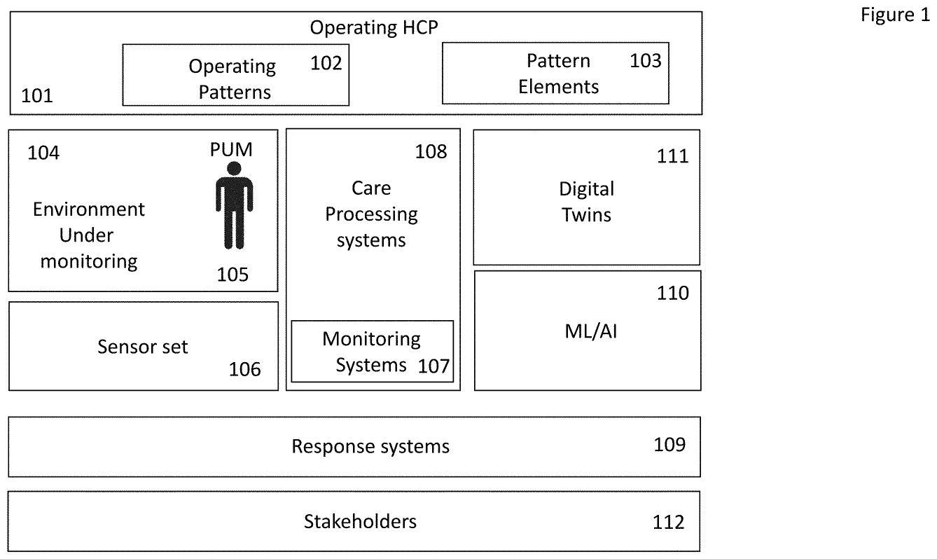

FIG. 1 illustrates an example set of modules that in combination provide, at least in part, the systems for the monitoring of a person under care described herein. Each of the elements of the figure are described herein. The person under monitoring (PUM-105) in an environment (104) which includes one or more sensors comprising a set of sensors (106), which are, at least in part, monitored by monitoring systems (107) in combination with Care processing (108) and the operating HCP (101), operating patterns (102) and pattern elements (103), in any arrangement, constitute the care and wellness monitoring of that PUM. This can include machine learning (110) and digital twins (111) in any arrangement. The care signals generated within such a system may be used by one or more response systems (109) to alert, communicate and/or instruct one or more stakeholders (112) to undertake an action is support of the care and wellness of the PUM.

Care Processing in Context

Current signal processing is predominately data centric, in that the data generated by the sensor is evaluated by a process designed to extract signal from that data. Generally signal processing is not contextual, in that even where it may include feature extraction this is focused on the data generated rather than the context in which that data is generated. Many systems use weightings and other forms of data metrication to evaluate the incoming data streams, often to identify specific features of the data set, usually described as feature extraction.

Some sensors may be configured to undertake feature extraction, where specific feature sets, such as those used for image processing and other similar functions are employed. This can include detection of movement and the like. These feature sets are often incompatible across multiple sensors, as each sensor has a proprietary implementation and the result and output of the sensor may not include the originally captured data.

Currently many signal processing techniques involving multiple sensors often use data normalization to establish a common data set which can then be evaluated by further processing. One often used aspect is the use of time as the baseline for many signal processing techniques, where the incoming data set is evaluated on a time base, usually linear elapsed time expressed in appropriate units.

The approach described herein adopts a different strategy, whereby a pattern or pattern framework, specifically configured to represent the situations that are consistent with the person under monitoring (PUM), their environment and their health care profile (HCP) that represents their current care state, is used by the Care processing monitoring systems as the context for the evaluation and/or processing of the data generated by one or more sensors monitoring the environment and/or the PUM. In this manner the pattern or pattern framework may incorporate a diverse range of sensors whose data outputs have no common normalization.

In some embodiments, the recognition of the patterns generated by the one or more sensors, may include sequences of events and/or signals that occur over a period of time where that time may be not be sequential. Such events and event sequences may include data from one or more sensor, where a first sensor generates data that the Care processing identifies as a variation in the care and wellness state of a person under monitoring and either directly and/or in collaboration with the first sensor communicates a configuration variation to one or more other sensors so as to verify, validate and/or augment the data from the first sensor, so as to increase the efficiency and accuracy of the determination of the events and/or event sequence in pursuit of the identification and determination of one or more care signal representing the variation in the care and wellness state of the PUM.

The patterns or pattern frameworks deployed herein, can have a non-linear, non-sequential, asynchronous, quantized and/or other time basis, in that rather than capturing all data emitted by any set of sensors on a linear or sequential time base, the system can use an established quiescent state of at least one sensor set for an environment and incorporate one or more patterns for that environment, which can include the presence of a person being monitored for care (PUM), to evaluate any differences from that state as captured by one or more sensor. In this manner the data sets of the sensors can be evaluated in the context of the at least one pattern operating in the Care processing systems involved in the monitoring process.

This approach can include the use of nested, hierarchical, windowed, ordered or other arrangements of patterns such that the Care processing system may deploy at least one pattern as the primary Care processing monitoring pattern or pattern framework, with other patterns or pattern frameworks providing alternatives. These alternatives may be operating upon digital twins of the PUM and/or their environment in combination with one or more machine learning techniques. These patterns and/or frameworks can be exchanged dynamically, such that if the state of the environment changes and that change is consistent with more than one pattern or pattern framework, the monitoring system may use probability analytics to determine which pattern or pattern framework is primary and which others are secondary and/or alternates.

The contextualization of the data generated by one or more sensors in an environment involves care signal processing systems supporting that contextualization. This is achieved through the use of an overarching care framework, described herein as the Health Care Profile (HCP) which in turn includes a set of patterns that initially are exemplar for that HCP and using the data sets generated by the sensors become populated so as to be representative of the behavioral patterns of a PUM in an environment. This approach provides the Care processing with a context in which to evaluate data sets of any type and complexity in support of care and wellness provision for the PUM.

This can include the exchanging of patterns and pattern frameworks, within the overarching HCP, which may be dynamic and responsive to changes in the monitored environment, and in some embodiments a set of such patterns or pattern frameworks may have associated weightings, that are representative of the accuracy of that pattern or pattern framework to predict the likely outcomes in a monitored environment.

The specifications of the patterns may range from simple, for example monitoring occurrences, such as coughing, that are indicative of a PUM condition as expressed in their HCP, in this case breathing difficulties, to complex, such as where multiple sensor data is aggregated, for example where a PUM has multiple health conditions and/or has memory impairment. These patterns may be created from sensor data sets as the behaviors of a PUM are observed and potentially replicated from other PUM who have similar health conditions and/or behaviors and/or may be specified by one or more care village systems and/or authorized stakeholders.

In some embodiments there may be multiple patterns operating simultaneously, with the same or differing sensors providing data for each of these patterns.

One aspect of the system is the manner in which data from one or more sensors is interpreted. A single event, such a movement detection can be evaluated in the context of the pattern that is operational at that time. For example, if the pattern is “night sleep,” representing a person occupying a bedroom at night for the purpose of sleep, then the movement detection may be cached and when a use of water flow is detected and a second movement detection is generated, an event, which may be represented as a token, representing a use of the bathroom at night may be created and stored.

However, if the movement detection does not have the other sensor data generated, then at least one further pattern may be invoked, for example awake at night pattern may be invoked, which can include configuring other sensors, such as smart light bulbs and the like to provide data that indicates the person is active.

An aspect of the Care processing is the detection, identification and/or validation of a care signal which, at least in part, represents a state of the PUM that may require an action or response, including further sensing. These care signals can represent events and/or event sequences, which are representative, in whole or in part of behaviors of a PUM, that correspond to the care and wellness state of the PUM. The use of quiescent states of care and wellness of the PUM can provide Care processing with the context for the detection and identification of such care signals by Care processing systems.

For any one or more sensors, there is a quiescent state, from the perspective of the system monitoring the environment, for example a Care processing system, where the sensor is either not providing any data to the system or the there is no change in that data. Sensors can have state, in that they are operating and at least one of collecting, measuring, processing, storing and/or transmitting data to the systems that have configured the sensor and established the command and control of the sensor operations, such as a Care processing system. The data generated by these sensors provides a representation of the sensed behaviors of a PUM, and as such can represent these behaviors as patterns or pattern elements, which in turn have state, in that the data provided by the sensors, for example in the form of a multi-dimensional feature set, can represent a state, including the quiescent state of that behavior.

A Care processing system may evaluate the data sets represented by a multi-dimensional feature set so as to determine if one or more of the data sets represented by the dimensions have a variance that exceeds one or more thresholds or other specifications employed for evaluation. This can include multiple sensors data sets providing verification and/or validation of another sensor data set, to for example, reduce any false positives. In some embodiments, the Care processing may configure one or more of the sensors contributing data to the multi-dimensional feature set under evaluation, so as to provide verification and/or validation, increase the granularity of the data set and/or invoke one or more other characteristics of the sensor.

This can be evidenced by variations in the one or more behaviors being exhibited by the PUM and represented as such in relation to the one or more pattern elements and/or operating patterns. This can involve unique and specific data sets from one or more sensors which in isolation may not provide sufficient data to generate a care signal, however in aggregate with multiple sensors, the configuration of which is coordinated based on the pattern element representing the behavior of the PUM, such that the care signals are detected and identified. These care signals may be represented in the form of multi-dimensional feature sets, where a combination of sensor data expressed as those dimensions and the relationships between those dimensions form the specification of the care signal.

The Care processing monitoring system may configure one or more sensors in such an environment to increase the granularity, sensitivity and/or other configuration attributes of the capabilities of that and/or other proximate sensors, invoke other sensors from a passive to active state and/or undertake an action that requires a response from the monitored environment and/or the PUM and/or other stakeholder therein. This can include providing sensors with one or more configurations that vary the operative state and/or sensing capabilities of the sensor. In this manner the focus of the monitoring may be adjusted to establish which of the patterns or pattern frameworks most accurately represent the current and/or likely situation within the environment.

This can invoke further changes in the monitoring focus, such that other sensors, for example, a smart speaker is activated to determine the activity of the person, such as reading, getting a glass of water or food and the like, for example through monitoring the acoustic data of their activities and/or asking the PUM if they are OK, and what activity they are undertaking.

Pattern identification and determination may be done from one or more sensor set data sets, where such data sets can include complex sets of signals, events and/or data sets representing same. The identification of patterns can involve one or more machine learning systems that can be invoked, for example multi-layer neural networks. These networks may in turn be used to support potential pattern arrangements that can be evaluated in one or more digital twins of the environment and/or PUM under monitoring, such that the alignment of the sensor data sets and the behavior pattern data can be more accurate.

One aspect of the care village Care processing systems is the use of likely patterns for behaviors of a PUM that can have care and wellness impact as the framework in which sensor data is evaluated by the Care processing systems. For example, if a PUM is exhibiting behavior where they continually bump into furniture, this may indicate, in addition to the condition for which they are being monitored, that they are having vision problems. In this example, the Care processing systems may operate the two patterns, the original condition pattern and the vision impairment pattern to align the monitoring with the behavior of the PUM. Having established that the patterns match the behaviors of the PUM, then Care processing may generate an alert to one or more stakeholders indicating that the PUM may need vision correction and/or assistance, for example as new glasses with a more powerful prescription. However, the data and pattern may also indicate that their current medical prescription regime is causing the issues.

In some embodiments, a dataset of the physical attributes of an environment and/or the PUM may be used to establish baseline data for one or more pattern. This can include establishing the state of the environment and/or PUM, especially in relation to the quiescent state of an environment and/or PUM. Such data sets can include relationships between environments and stakeholders, including one or more PUM.

The determination of an optimum data set to be collected from a set of sensors, where each sensor has multiple capabilities such that only specific capabilities are selected and the attributes of those capabilities, such as time/duration/signal resolution/data type/data size and the like, can in some embodiments, be configured to conform to one or more pattern specifications. This can include selection of a specific sensor in a multi-sensor device, for example a smart phone, where the configuration of that sensor may be varied by the Care processing systems, such as when monitoring focus is changed, for example for verification and/or validation of an event detected by one or more other sensor that is providing data to one or more operating pattern. For example, the focus and zoom of a camera in a smart phone may be varied to verify an event that is provided to Care processing by another sensor, for example an acoustic sensor. situation

In some embodiments, care signal processing system modules can operate as part of a set of pattern frameworks to configure an available set of sensors. The data from these sensors can be held in a repository, such as an elastic repository, for a period of time, that is determined by the pattern framework specifications and may form a reference set of data. This data can be used to establish, for example, the quiescent state of an environment, which may include the presence of a PUM.

Each of these data sets can be sampled on a random basis to determine whether the data is within the specifications of the quiescent state of the pattern specifications invoked for that environment at that time. The rate of sampling, sample size and evaluation processing may be varied according the specifications of the quiescent pattern specifications. In some embodiments, reference sets may be used to establish thresholds and sample rates appropriate for the situation being monitored.

In some embodiments at least one sensor may be configured to be an edge sensor, where the data set is processed and/or evaluated within the sensor device or at a connected device physically close to the sensor on a real time, near real time or event driven basis. In some embodiments, this processing can be undertaken remotely in the cloud, however this is subject to appropriate communications being available. In some embodiments, this processing may be undertaken on the device that includes the sensor, where that device includes one or more communications capabilities, for example wireless cell coverage, such as 5G. In some embodiments the edge sensors may be connect to a care hub, or other similar hub or router device that incorporates one or more communications capabilities, including for example, cell coverage, such as 5G, PSTN using copper wire, cable, fiber or other hard-wired connectivity. In some cases such a device may have multiple communications capabilities with fail over systems supporting the multiple communications capabilities. This edge sensor may provide the leading-edge detection that can then be complimented, verified and/or validated by other sensors that have an established and/or predetermined relationship with that sensor. For example, a Micro-electromechanical systems (MEMS) microphone may be configured to listen for low frequency signals that are processed and evaluated at the edge sensor to detect events, such as footfall, and as such when such is detected, for example at night when a nighttime sleep pattern is operating, may communicate with other sensors, such as smart light bulbs or other sensors with active sensing, such as Frequency-Modulated Continuous Wave (FMCW) radar capability to determine the location, breathing or other aspects of the person.

An edge sensor may be configured, depending on the capabilities of the sensor to detect events and event sequences that could indicate a change of state of an environment and/or the PUM. This can include, for example, measurement of movements, such as footfall, gait, jerkiness, sudden movement and the like as indicators of a change in the mobility of a PUM, distinctive changes in timing, for example dwell time in kitchen, bathroom or other locations, indicating an activity that is taking more time than usual, changes in behaviors, including, over or under usage, consumption or other variances of activities that are part of a quiescent state.

The Care processing systems operate one or more pattern, each of which includes one specified edge sensor generating data that can then be processed so as to compare data with the quiescent state pattern data for that environment and/or PUM, including portions thereof

This approach of pattern determination, whereby the complete environment and the PUM are considered as a set of states, based at least in part on a quiescent state, that is created from a framework of both the environment and the PUM, represented as a set of patterns that include the behaviors of the combination of environment and PUM to form a data set for a Care processing system. The care processing system can collect the data generated by individual sensors, however the use of one or more patterns significantly reduces the amount of data processing required to identify those signals that indicate a potential or actual care incident. This approach enables edge devices, such as sensors, hubs, wearables and the like to undertake processing of such data sets at the edge. Such patterns may be operated on the device or sensors embedded and/or located in the environment, on specialized and/or standard off the shelf devices and/or other hardware in proximity to the environment being monitored. In some embodiments such sensors, devices and/or hardware may act as aggregators for data and patterns, located at the environment and/or remotely, such as in the cloud and/or in a remote hosting system, cloud services or other networked system, in any arrangement.

In some embodiments each sensor may include access to a repository where any data from the sensor is stored. Such a repository may be an elastic repository enabling the storage of data sets for a period of time that is, in part, determined by the pattern being operated. These repositories are described as elastic repositories. This data may be made available to care processing systems after an event or event sequence has been detected, and may be processed to identify characteristics of the data that were preemptive in relation to the identified event. This process may be undertaken across a number of sensors, using for example, machine learning techniques, and may then be incorporated into existing or new patterns for future deployment.

The care processing for care system is configured to use a set of patterns that are representative of the behaviors of the PUM in context rather than simply gathering all the data from all sensors. This approach involves the separation of the steady state background sensor data, representing the quiescent state, from those behavioral elements that are the context of the PUM as they journey through their respective HCP.

One aspect of the system is the at least one device which is configured to provide event and/or event sequence data sets to one or more care signal processing system. A sensor may be configured as the edge sensor in a dynamic manner, for example an FMCW sensor may be so configured in a living area and an acoustic sensor may be so configured in a bathroom. This dynamic transfer of edge capabilities may incorporate further sensors which have their configuration, including activation, deactivation, fidelity and/or other operating characteristics varied as part of an operating pattern and/or in response to data processed by the care processing system from at least one edge sensor. The configuration of each device may be determined, in whole or in part, by the care processing system, which can include devices, including sets of devices, with prearranged and/or dynamic relationships to each other, that can be configured to send events and/or event sequences, some of which may be in form of alerts, to other system elements, devices and/or stakeholders, including the PUM. This can include configurations to send aggregated and/or combined signals to a larger or other arrangement of local/edge/remote devices. Such configuration may be dynamically varied in response to observed conditions, patterns, events and data sets.

Within this configuration one or more sensor can be configured to optimize the output of such sensors, for example increasing the fidelity of the sensor, so as to detect or confirm, including validation and/or verification, of an event and/or event sequence. For example, this can include optimization of a MEMs microphone or other acoustic sensor and/or an active emission sensors, such as a FMCW device, to detect whether an immobile PUM is breathing and how regular that breathing may be. This can indicate whether the PUM is, for example exhibiting sleep apnea or other breathing related issues.

In some embodiments, data from individual and/or sets of sensors may be verified and/or validated by data from other sensors that are involved in monitoring the PUM and their environment. This can be the situation where, for example, multi-sensors devices, such as a smart-phones, smart watches or similar provide a set of data that can represent an event. This data from a single device may indicate a fall or other care or wellness event, and as such could trigger, for example, emergency or other responders. However, the care processing systems can receive further data sets from other sensors in the environment, for example, acoustic, camera, haptic, FMCW or other active emission sensors and the like and as such can validate and/or verify the data set provided by the single device. This data verification and/or validation can occur within the pattern being monitored at the time, and depending on the event and the verification and/or validation, may indicate that a transition to another pattern is taking place. This approach reduces the propensity of single device and/or single sensors data sets to indicate an event that results in a false positive. Which can result in unnecessary escalation of the event that results in EMT or other resources being deployed, when in fact they are not required. The verification and/or validation may be undertaken by the care processing on a sensor by sensor basis, and in some embodiments the outcome of this processing may be stored and used in differing PUM and environment situations as well as providing training and/or comparison data for machine learning systems.

In some embodiments, care processing systems may be distributed across multiple sensors, devices, hubs and/or other hardware. This can include the use of feature recognition and other techniques that are resident and operating on, for example, sensors, devices and/or hubs, such that data generated by a sensor may have undergone processing to extract one or more features from the data captured by the sensor. For example, if a camera sensor is configured to capture edge features of the images being monitored, this data can be communicated to the care processing system, if and when edges that are consistent with a PUM, move form vertical to horizontal. In some embodiments the raw data feed may be stored in an elastic repository, for example for a period of time that is representative of human behaviors being monitored, for example 5 minutes, 30 minutes one hour or more and the like, and simultaneously the sensor is processing the incoming data to extract edge features which are then communicated to a care processing system. For example, the care processing system may then, on receiving a data set processed by the sensor, where the data indicates a change in the orientation of the PUM, may then active other sensors to confirm this change and instruct the elastic repository to mark the data held from the original sensor for some degree of persistence such that the event under consideration may be investigated. For example, if the orientation change was due to a fall, the camera data may be made available to one or more other stakeholders and/or further care processing systems.

The use of distributed care processing across multiple sensors, devices and/or systems, including care hubs, supports the privacy of the PUM within their environment, whilst providing an effective monitoring of their care and wellness. As each sensor can have the capability to process the data received by that sensor, using for example, feature extraction, that sensor may communicate only the extracted feature to a monitoring system whilst simultaneously storing the raw data in a repository. In some embodiments, this communication may be in the form of a token. This enables the monitoring systems to determine whether the feature set in comparison to the operating pattern, matches or satisfies the criteria of a care and/or wellness event, whilst maintain the privacy of the PUM through non-disclosure of the raw data. In some embodiments, such raw data may be made available to authorized and authenticated stakeholders, such as medical professions, EMT, emergency responders and the like.

One aspect of the processing of the data generated by the one or more sensors, devices and/or systems, is the use of distributed processing across multiple processing capabilities. For example, this can include processing on the sensor and/or device, which may include for example, feature identification, categorization and/or extraction and the like. In some embodiment such sensors and/or devices may have access to additional processing capabilities, such as local care hubs and/or other co-located and/or remote, for example cloud based, systems.

A further aspect is the deployment of distributed decision systems where the configuration of one or more sensors may be determined by one or more other sensor and that configuration may be part of a decision process that is initiated by one or more modules, devices and/or systems, for example a care hub. For example, if a monitoring focus is increased in response to a variation in an operating pattern and/or pattern element, the configuration of a sensor by, for example a care hub, to increase fidelity, accuracy and/or timing of that sensors operations, including for example employing feature extraction, identification and/or recognition, that configuration change may instigate further configuration changes in other collocated, logically or physically sensors, so that the data set of the first sensor is enhanced, including being validated, verified or otherwise confirmed, by those other sensors in support of an aggregated data set that is responsive to the initial decision processing of the instigating module, device and/or system, for example a care hub.

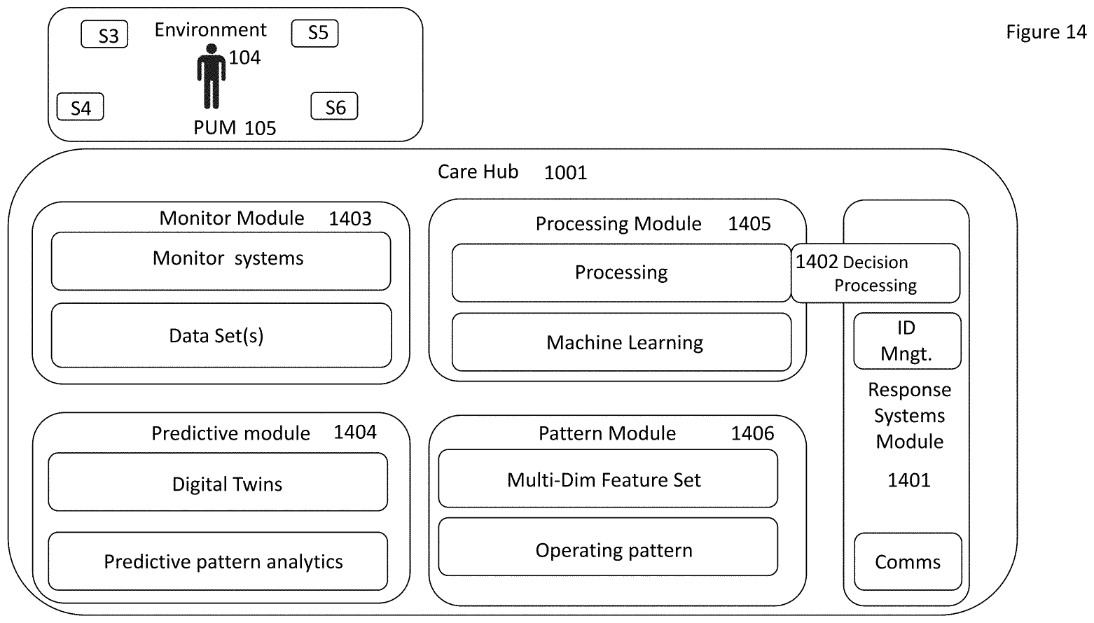

FIG. 14 illustrates an embodiment of a care hub (1001) that is employed as part of the monitoring of a PUM (105) in an environment (104), comprising a monitor module (1403), processing module (1405), predictive module (1404), pattern module (1406), decision processing module (1402) and response systems module (1401) all of which may include one or more sub modules, by reference or embedding which may be local or remote. For example, a sub module may be included in a care hub as a hardware instantiation, including for example protected processing, secure encrypted storage and hardened identity, processing, key management and other security features to ensure that confidential information, including communications, is protected.

Such an approach can include distributed decision processing that identifies that one or more sensor is operating in an incorrect or faulty mode, and as such may be reset, reconfigured and/or the data set generated may be disregarded or have one or more attributes assigned that attest to the fault condition. Such condition may then be reported to one or more systems for fault management.

In some embodiments byzantine algorithms and/or consensus algorithms including similar approaches may be employed for both identification and/or configuration of such sensors in any arrangement.

In some embodiments, tokens may be exchanged between sensors and/or devices that are operating in a quiescent or other operating state where each token, may through reference or embedding, including the token itself as an instance of such operating pattern and/or pattern element state information, and may though this exchange of tokens between such sensors and/or devices can maintain this state across multiple sensors and/or devices in an environment. For example, a token may include configuration specifications for one or more sensors and/or devices, such that those specifications that have been disseminated by one or more decision processing process, including those involving the sensors and/or devices themselves.

This use of tokens may support the privacy and confidentiality of information communicated among and the sensors, devices, systems and modules comprising the monitoring systems in an environment.

In some embodiments, a pattern can be determined in context with identification and transmission through the use of tokenized instantiations of such patterns.

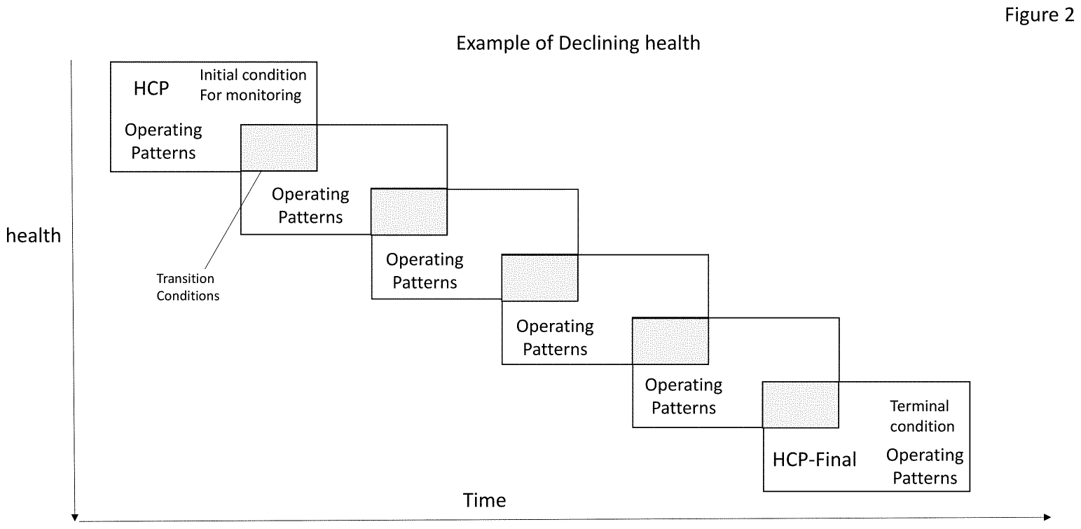

FIG. 2 illustrates an HCP representing the care journey of a PUM from their initial care state, which represents the initial care condition for monitoring, through a series of care states that lead to a palliative hospice care condition and ultimately a terminal care condition. The care path illustrated here may not be linear, nor may the HCP states illustrated herein be of the same duration, have the same transition conditions or have care condition declines of the same severity, rather this care journey, for an individual PUM, is likely to be unique to that person. However, the HCP commonalities across many PUM with the same conditions can be evaluated for patterns and behaviors that are evident, at least in part to those HCP conditions.

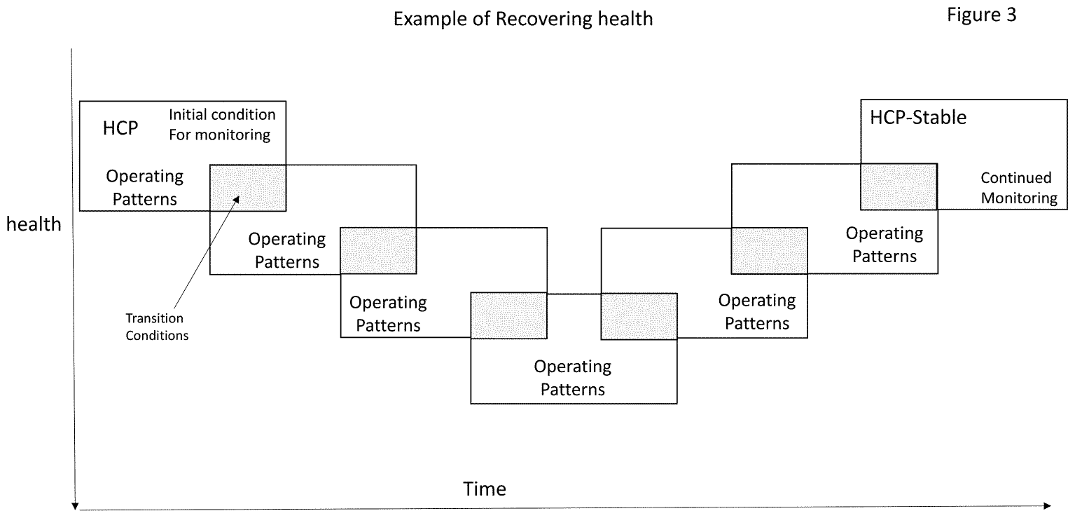

FIG. 3 illustrates a further HCP, where the PUM makes a recovery to at least the initial condition that caused them to be placed under monitoring. Such a situation may, for example, be part of the HCP journey of a PUM as illustrated in FIG. 2.

Behavior Patterns

In most all environments, such as a domestic environment or care facility, where a PUM is domiciled, the behaviors of a person within that environment exhibits certain patterns. For example, the use of kitchen and bathroom facilities can have certain timing for use, with dwell time in each being within certain parameters. Further examples include, bedrooms, kitchens and living areas where there can be consistent dwell times for such activities as sleeping, cooking, watching TV, reading, researching the internet and the like.

One aspect of the behaviors is the monitoring of the activity and dwell times to establish a pattern for the movement of a person in an environment. This can include the monitoring of entry and exit information for a particular area, for example bathroom, kitchen and the like, as well as movement between these differing functional areas.

One challenge for all care processing is the recognition of a change in state of the input being monitored, where that state change is an indicator of an event that is occurring or could be forthcoming. In the HCP environment for example this may include recognition that a user is tripping on an existing edge in their floor, or a piece of furniture, such as a couch, causing at one or more sensor to store this data. For example, a sensor incorporated within a device such as an accelerometer, and/or an acoustic monitoring device, camera and or the like.

In this example these devices may store the data and have that data polled by an edge device or other system monitoring process. The evaluation of this data may be undertaken within a pattern framework, where a known set of precursors to an event, such as a fall is an increase in the number and rate of missteps a person may take in their environment.

In such an example the system may be configured to alert a care taker, family member, neighbor or other stakeholder of this occurrence, so that remedial action may be undertaken to avoid the likely fall. The system may calculate the probability of the fall from this data set and advise the person in the environment to cease or reduce their movements until help can arrive. This advice may be communicated through a carried or wearable device, a smart speaker, smart TV or other suitable device in the environment.

Such an example situation, may include activation of one or more other sensors, such that they are configured to observe the person and the environment in more detail with an increased monitoring focus. This can also include the configuration and activation of devices that provide medical or other health monitoring of the person and environment, such as blood pressure monitors, temperature and climate control and the like.

This can include configuration of the devices to monitor the environment in a manner that is aligned with the events being monitored. This can include using differing arrangements of devices and sensors with differing configurations to create data sets that are suitable for the system to undertake evaluation and/or to be transmitted in an appropriate format to one or more stakeholders. The relationship of this data set to the environment and the person being monitored (PUM) may cause the system to invoke different patterns and pattern frameworks in response, such as for example those that may be required prior to or on the arrival of a care taker, medical and/or response team and/or the like.

Where the data set is sufficiently aligned with a preformatted event sequence response arrangement and/or is within a median deviation or other threshold to a predictive model, this may cause the system to invoke one or more patterns in response to the data set and the situation that it represents. This can include the matching of detected patterns of behavior to pre-configured response arrangements.

Codification of Behaviors as Patterns

Certain behavioral characteristics forming at least on pattern may be monitored and that behavior, sequence of behaviors, event or sequence of events in any arrangement may be matched in whole or in part to a pattern of such behaviors and/or events that is stored by the system. These arrangements can include hierarchical, sequential, dependent and/or the like.

These stored patterns may in turn have response arrangements, that in whole or in part, are responsive to these identified monitored behavior and event patterns. This can include sets of configurations that are deployed to sensors in the environment in response to data from one or more sensor. In some embodiments specifications may be stored and invoked when certain behaviors are exhibited and/or match one or more stored patterns. This can include events and alerts to one or more stakeholders and/or other systems that may then provide one or more response.

The matching of the monitored pattern to the stored pattern may yield varying degrees of certainty as to the match of these patterns. For example, a pattern may match 6 out of 7 behaviors and 4 out of 5 events in a time period common to both patterns. This may produce a pattern matching matrix where the system may invoke further care processing and/or configure further sensors to verify and/or validate such pattern matching.

In some embodiments, the determination of recurrent behaviors, such as where a person regularly sits, when they prepare food, use the bathroom, go for a walk and the like can be identified as pattern elements as they represent, at least in part, the routine behaviors of a PUM. Such elements may represent part of the quiescent state of a pattern, where for example the recurring occurrences form a sequence of PUM behavior. In some embodiments these recurring behaviors may be designated as pattern elements. For example, a behavioral change in one or more of these recurring behaviors and/or of the sequence of such behaviors, may represent an indicator of a transitional state, such that the PUM is transitioning from one pattern to another. This can include situations where the pattern to which the PUM is transitioning may be one of a number of potential other patterns In this example, a digital twin may be used to spawn additional instances of each of the likely patterns that the PUM may be transition to. The care processing may then deploy and/or create a configuration for sensors that can be used to verify, validate/or inform as to the most likely pattern candidates, so as to optimize the monitoring and/or detection of the pattern and the behaviors represented thereby. This approach enables the determination of which pattern(s) best matches the situation.

In an example where there are two patterns with equal likelihood, the care processing may configure the sensors so as to provide sufficient data to both patterns to detect at the earliest possible moment which pattern best represents the actual events unfolding. This can include creating alerts, messages and/or other data sets to be transmitted to appropriate stakeholders and/or other systems, and may also include certain pre-configuration, such as determining the locations of specific stakeholders in relation to the PUM and, for example calculating timing and other metrics in support of care of the PUM.

In some embodiments, an edge device and/or other sensors may have their data output directed so as to match a set of pattern elements. Each pattern fragment is a part of a pattern framework and/or a pattern all of which are a set of patterns that are part of an HCP or where the patterns indicate a transition is likely between one HCP and another across two HCP. The deployment and operations of these fragments may be managed by the care processing module and may be operated on the sensors and devices embedded in an environment and/or on the digital twins of such arrangements.

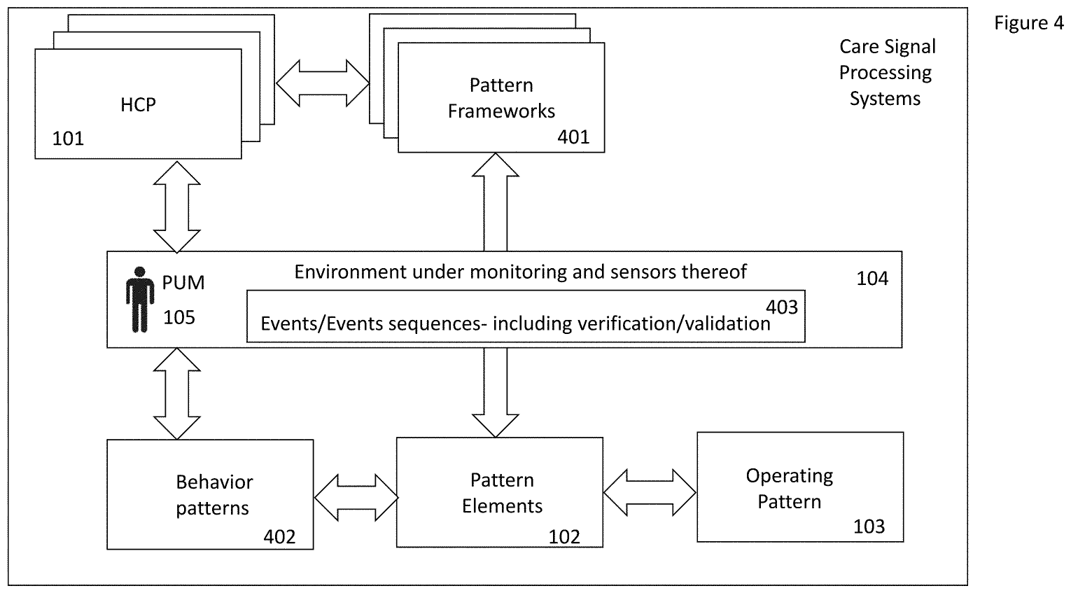

FIG. 4 illustrates a set of modules for monitoring a PUM (105) in an environment, where the combination of HCP (101), behavior patterns (402) and the elements thereof, for example pattern frameworks (401) and the pattern elements (102) can combine to form a care signal processing systems which can include configuration and relevant command and control features to support the effective monitoring of a PUM (105) within the context of an HCP (101). This can include the environment in which the PUM is being monitored (104), in which one or more events, including sequences thereof (403) may occur. In some embodiments behavior patterns (402) may be represented by one or more pattern elements (102), which in turn may form, in whole or in part, an operating pattern (103). In some embodiments, operating or other patterns may form pattern elements of, for example, a further operating pattern, such as in a hierarchical manner.

One aspect of the system is the use of multi-dimensional feature sets as representations, in whole or in part of a behavior pattern of a PUM expressed as a pattern or pattern element. These feature sets comprise multiple sensor data sets that include relationships between those data sets from the one or more sensors embedded in an environment.

These data sets can be represented using, for example manifolds, Hilbert spaces or other representations capable of storing each individual data set from a sensor and the relationship of that data with data from another sensor. This relationship can comprise data sets from multiple sensors, for example a temperature sensor, acoustic sensor and motion detector, where the relationship, for example when a PUM is sleeping represents an at rest or quiescent state is represented by an operating pattern, for example the night sleep pattern.

These relationships can form feature sets that are representations of the aggregate data of the one or more sensors, for example represented as multi-dimensional feature sets, such that the features are defined as the relationships between the data sets of the multiple sensors. Such relationships may be expressed, for example as ratio's, functions and/or algorithms, spatial and/or graph-based expressions and/or the like. In this manner a feature set representing relationships between two or more sensors can be used to determine the state of the PUM in an environment. Such a relationship may include one or more thresholds, variances or other data sets to accommodate sensor data variations. For example, the relationship between acceleration in three axis and the location, height and posture of the PUM can be evaluated to determine if a fall or a minor trip has occurred. These sensor data sets may have further relationships with sensors for detection of audio, visual, breathing, heart rate or other sensed data sets. The combined evaluation of these data sets in the form of a multi-dimensional feature set can include both sequential, for example an event sequence as represented by the sensor data sets and/or in parallel.

Such feature set evaluation can be used to detect transitions from one pattern or pattern element to another, as the sets of relationships represented by the multi-dimensional feature set can provide a framework in which individual sensor data set variations can be evaluated, at least in part through their relationship to each other and their correlation to the monitored behaviors of the PUM that such feature sets represent.

As each sensors data set may vary, the utility of these variations as a metric for the evaluation of an alert, event or response is limited, including by the capabilities of the sensor, even though that sensor may include and/or have access to feature set identification and/or processing. Whereas variations exhibited by the combined feature sets of multiple sensors, especially in the relationships of one or more sensor to other one or more other sensors can provide a more accurate and comprehensive representation of the unfolding circumstances of a PUM. These relationship changes can form indicators that a PUM is transitioning from one operating pattern to another.

In some embodiments, each pattern or pattern element comprises a composite of data from one or more sensor representing the behaviors of a PUM in an environment.

Feature sets can comprise multiple dimensions, where each of the sensor data can form, in whole or in part, a dimension of the feature set. These dimensions can be represented in one or more multi-dimensional feature sets

One aspect of PUM behavior is the routines of daily life, including for example, sleeping, eating, bathroom use, exercise, entertainment, social and the like. One aspect of the cultural behaviors, such as broadcasts, such as TV and radio, internet, including streaming and interactive and other content and the like. As with many other human behaviors, the timing, selection, duration and other media or cultural behaviors may contribute to and/or in whole or in part, form patterns and/or pattern elements representing behaviors of the PUM and/or other stakeholders.

For example, a PUM's digital patterns such as watching Netflix or other streaming services and/or their internet searching may be indicatory and/or revealing of changes in their health and wellness state. However, such information can be highly revealing as to the PUM and/or other stakeholders and as such this PII, may represent a significant privacy risk if it becomes widely available.

In some embodiments a care hub may act as an aggregator for one or more sensors that are involved in monitoring the digital interactions of a PUM, so as to monitor patterns and/or pattern elements representing that behavior. In this manner the data may be evaluated to determine any care and wellness impacts, whilst protecting the privacy of the PUM and/or other stakeholders through encryption of the data and limitation of the distribution of the data. This can include deletion of the source data after the patterns have been extracted and/or identified and may include the use of tokens to represent such data, patterns and/or pattern elements in any arrangement.

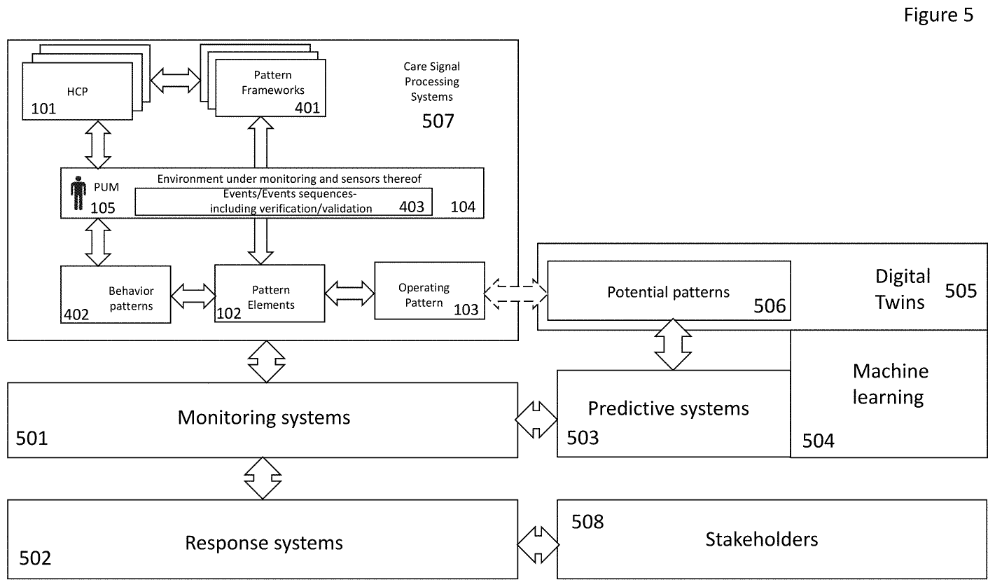

FIG. 5 illustrates the care processing systems (507) integrations with a set of response systems (502), which in turn are integrated with the appropriate stakeholders (508) for that PUM (105). This can involve such stakeholders (508) as emergency responders and systems, care taker's, family, neighbors, friends, medical professions and the like in any arrangement. The responses may be derived in part from one or more specifications of the care condition state represented by the care processing systems and may, for example, include the configuration of the sensors with differing monitoring focus for the differing stakeholders in any arrangement. The monitoring systems (501) are integrated with the environment monitoring sensors (104) and the response systems (502). The monitoring systems can interoperate with one or more predictive systems (503), machine leaning modules (504) and digital twins (505) which may in part determine potential patterns (506) and/or pattern elements that can be instantiated, in whole or in part as operating patterns (103).

Reference Data Sets

To establish the baseline for effective processing of multiple sensor data sets in an environment, a reference data set is created for those sensors individually and in combination. One key aspect of the system is establishing the “at rest” state of a sensor in an environment. This involves configuration of the sensors so as to have a rest or quiescent state that incorporates the sensor measuring the environment when there is no activity. As such each sensor generates a data set which can become part of the reference data set for an environment.

The reference data set can have state, such as with a person under monitoring (PUM) present, an activity being undertaken, for example sleep, watching TV, eating, self-care and the like and/or other redefined or metricated data sets. These data set may form pattern elements and/or represent recurring behaviors for a PUM.

One aspect is the integration of data sets from differing devices, for example a sensor measuring temperature and another capturing acoustic signals. The integration of these data sets is typically undertaken by normalization, however if the metrics used for each sensor are sufficiently different and have no effective equivalence, then the integration is undertaken in the context of a pattern and/or pattern elements representing exhibited behavior. These are described herein as behavior patterns.

In this manner disparate data sets may be integrated by the care signal processing systems to provide a consistent reliable measure of the state of the environment in relation to one or more pattern that integrates the individual sensor data sets. However, the determination of every possible combination of data sets into integrated sets and patterns is likely to be intensive and always has the N+1 problem, in that there is the possibility of one pattern that is not yet identified. To resolve this the system uses the reference data sets in combination with the environment specifications, for example, in the form of digital twins, in combination with machine learning for the identification, classification and/or storage of these additional patterns.

The integration may be represented using a number of techniques, including for example, graph databases, Hilbert spaces, Reiman or other manifolds, where the individual data sets from one or more sensors is expressed as a relationship to another sensor data set. For example, if a reference data set represents a PUM undertaking a recurring behavior, where the date for that behavior is within any thresholds of the quiescent state of that recurring behavior, for example a pattern element, then the individual sensor data, expressed in the metrics of that sensor can have a relationship with another sensor data set monitoring that same recurring behavior at the same time, such that the relationship between the potentially disparate metrics of the sensors comprises a metric for that quiescent state. This can include multi-dimensional representation of the environment and the behavior of the PUM within that environment such that the pattern or pattern elements provide sufficient metrics so that an alert, event and/or response system may determine that one or more relationships between the sensor data sets has, or is likely to, breach one or more thresholds that the processing system has been configured to represent the state of that environment and PUM.

The expression of these threshold conditions and associated configurations can involve use of digital twins and machine leaning, separately and in combination, so as to determine the probability of the state of a pattern or pattern elements changing. This can include dynamic adjustment of that configuration of thresholds and any response systems response arrangements specifications in regard of the prevailing conditions. For example, if the external temperature is excessively hot or cold, the configuration may vary the one or more thresholds in light of a changed behavior of a PUM, for example adding or removing clothing, shifting positions, changing HVAC settings and the like.

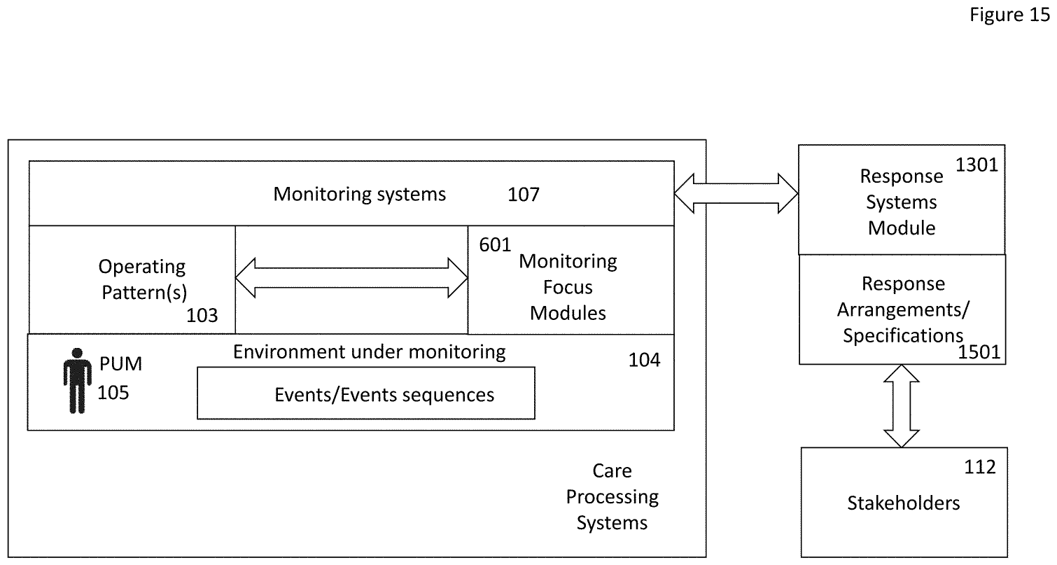

FIG. 15 Illustrates a PUM (105) in an environment (104), where one or more operating patterns (103) are unfolding, and in conjunction with monitoring systems (107) and monitoring focus module (601), a wellness care state is identified that invokes response systems (1301), which may have predetermined and/or dynamically created and/or varied response arrangement specifications (1501) that are employed, resulting in an appropriate response being undertaken by one or more stakeholders (112)

In some embodiments, this reference data set can be a snapshot of the state of an environment and the PUM therein and can comprise data generated by each sensor individually and/or in aggregate in any arrangement. This can also include data sets that are accumulated over one or more time periods. This can include establishing the quiescent state of one or more sensors in that environment. Such snapshots may be persisted and used, in whole or in part, as a corpus for one or more machine learning system.

This may also include specified relationships and configurations of a specific sensor with other sensors such that combinations of sensors and configurations provide an aggregate capability, for example one that is focused on a specific PUM behavior including patterns and/or pattern elements. These configurations may enable these sensor sets to operate at differing granularities and resolutions so as to preserve the privacy of the PUM in circumstances such as when the state of the PUM and environment is quiescent.

There are some data relationships that have well understood parameters, for example those that represent the laws of physics and other measurable outcomes. These relationships may be defined as algorithms and used in typical configuration and data normalization processing.

The care processing system operates a set of patterns into which the data sets being generated by the one or more sensor in an environment is integrated. This can involve one or more pattern being determined as operating at that point in time. However, a further set of patterns can integrate the same data sets into other patterns which can then be evaluated to determine the most likely representation of the situation occurring in an environment at that time. This processing can be undertaken through the use of digital twins in combination with machine learning systems.

The use of reference sets which represent the state of the environment, the PUM and the behaviors operating at that point in time can provide a framework in which this processing and evaluation occurs.

Reference datasets may need to be updated as the environment changes, the PUM's condition evolves (recovery, decline, aging, learning, etc.) and/or as sensors get added, removed, updated and/or replaced.

In some embodiments reference patterns and pattern frameworks may incorporate medical diagnosis information, such as that commonly used by the medical profession to identify specific health and care diagnosis. This may include specific thresholds, metrics, behaviors or other exhibited traits of a person under care monitoring in an environment. In this manner a health professional may be able to monitor a person for certain behaviors and characteristics and when such are identified by the system receive alerts or data sets. In some cases this may include alerting other stakeholders involved in the care of the person and potentially invoking actions and responses by those stakeholders.

In some embodiments, care processing system may use sampling techniques for data generated by sensors, if the system state is quiescent. Such an approach may increase efficiency and privacy.

Monitoring Focus

A care processing system can operate as part of a monitoring control system that can configure and control each of the sensors and/or devices in an environment and provide and/or support the resources, such as devices, sensors, computing, storage, machine learning, algorithms and the like to enable this functionality.

The monitoring focus system, in some embodiments, forms part of the care processing system and provides a dynamic ability to vary, within the capabilities of each individual sensor and/or aggregations thereof, the environments overall sensing capability so as to focus on one or more aspects of the environment and the PUM. This can include the aggregation and accumulation of data from multiple sensors to form integrated patterns that can provide a more detailed data set of the environment and PUM, which may include multi-dimensional feature sets

A further aspect is the delegation of configuration of a monitoring focus module to authorized and authenticated stakeholders, such as for example medical professionals, emergency response teams, care stakeholders and the like.

One aspect of the system is the distributed nature of the configuration of the sensors in that with a sensor having at least one relationship with another sensor, the first sensor may configure the second sensor to undertake a more detailed, focused, granular, higher resolution or other configured operation, so as to generate a data set that, in combination with the initial data set from the first sensor, comprises a more complete, accurate and/or informing data set. This can include each of the sensors increasing the volume, quality of other attributes of data and the like they are generating, which may be represented as the dimensions of their contribution of a multi-dimensional feature set.

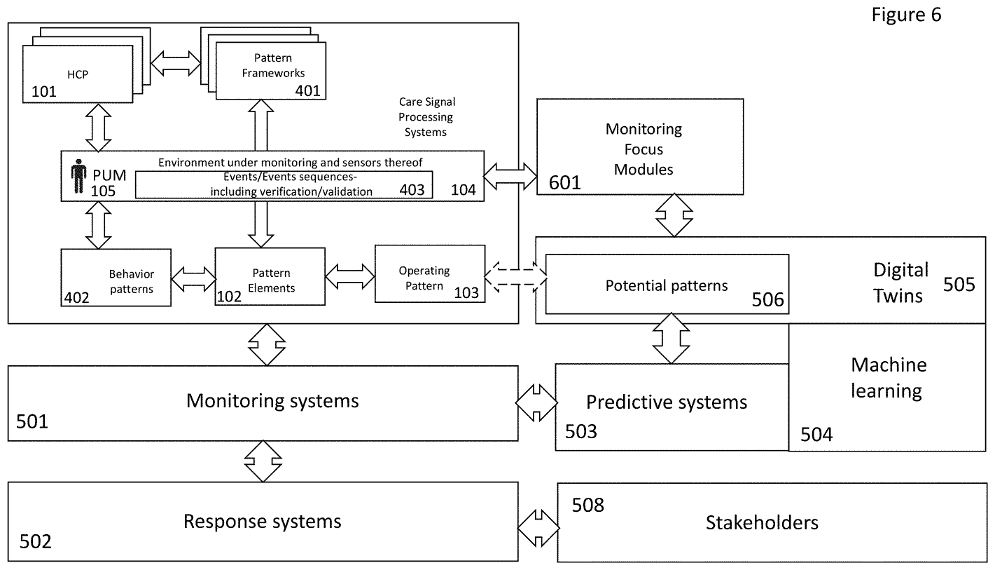

In FIG. 6 Monitoring focus modules (601), interoperate with environment under monitoring (104) which can include pattern elements (102) and operating patterns (103) as well as predictive systems (503), machine learning (504), potential patterns (506) and digital twins (505) in any arrangement.

In some embodiments at one or more digital twin can be operating, in which one or more pattern is operating, configured at least in part by the sensor data from the currently deployed operating pattern of the monitoring system. In combination with application of one or more machine learning techniques, this can result in detection of one or more features that hitherto have not been identified and/or classified. For example, this can include data from one or more sensor over an extended period of time, for example a period of time that exceeds that sensor's ability, including any accessible repository, to store that data. For example, this may be weeks or months, where the digital twin can provide the This can include data and/or configuration information from multiple sensors that have not yet been combined, aggregated and/or evaluated as a set by a care processing system. These newly identified features can be represented by further dimensions that can be integrated into existing multi-dimensional feature sets and/or may be represented as dimensions in new feature sets. In some embodiments, the system may predict and postulate that the data from one or more sensor may be represented as a feature of that data, for example an event, action or other identifiable attribute. In this manner such a feature may be passed to the operating pattern and using one or more sensor, be evaluated for accuracy in that sensors' operations, including supporting data from other co-located sensors with which the first sensor has or establishes relationships. These features may also be created through extrapolation and/or interpolation of data sets from situations that are considered to have sufficient equivalence, as determined in part, by one or more machine learning and/or statistical techniques. In this way the experiences of a PUM in an environment may be correlated to other PUM in other environments, where the feature set, including multi-dimensional feature sets and their respective representations, can provide further informing data for that situation.

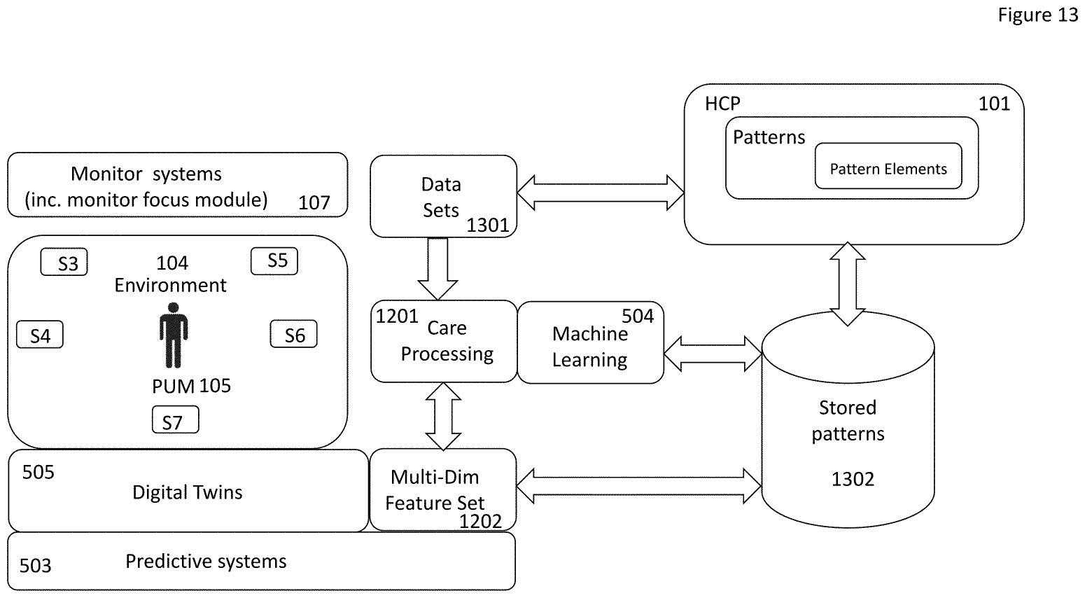

FIG. 13 illustrates a PUM (105) in an environment (104) where sensors (S3 through S7) generate data sets (1301) that can be processed by care processing (1201) to form, at least in part, multi-dimensional feature sets (1202), which may then be used by digital twins (505) and predictive systems (503) to create, augment, modify and/or validate stored patterns (1302) that can be employed by HCP (101).

Such features may also be used by a care processing systems to validate and/or verify existing data to, for example, confirm the trajectory of a care condition of a PUM in an environment and/or to vary the configuration of a sensor set and/or care processing systems for the purpose of care management. This can include the configuration of care processing, sensor and/or stakeholder arrangements to mitigate a predicted or anticipated care conditions.

In some embodiments, a device in an environment may have an executive function, such as an override that enables that device to be configured to become active with at least one sensor of the device, where that sensor may be calibrated to provide a data set within the capabilities of the sensor and device. This configuration and control may be undertaken in an emergency situation, where both the device and the system have a pre-agreed specification, for example instantiated through a common API, as to the declaration of an emergency. In some embodiments this can involve the system and the device exchanging tokens comprising embedded and/or referenced information. Such an exchange can include one or more identity, authentication, authorization and/or access control specifications and/or enforcement mechanisms.

The combination of sensors communicating with other sensors to integrate their data sets, where a first sensor matches a data set to at least a part of a pattern and then communicates with a second sensor in the same environment, configuring that sensor to provide data already collected and/or begin collecting data such that a care processing module may integrate both data sets with the intention to confirm or verify the first sensor data set as presenting an event, including part of an event sequence, that matches with a high degree of accuracy an element of a pattern, provides a rigorous and accurate determination of the situation occurring in an environment and the PUM domiciled therein. Such an approach may include the use of multi-dimensional feature sets which are used, in whole or in part, to evaluate the incoming data sets and to establish any changes in the operating pattern and/or pattern elements.

This combined information set may be provided to one or more operating digital twin of that environment and the PUM therein, to identify further data that can be expected from other sensors in that environment. This can involve the care processing module and/or monitoring systems changing the state and/or configurations of such other sensors, so that the unfolding events may be accurately determined. This can result in further focusing of the monitoring capabilities of the environment and/or initiation of at least one response arrangement where the events indicate a care incident of sufficient severity to warrant such response. This can include responses that are anticipatory, such as alerting a neighbor or other stakeholder to assist a PUM and the like.

A sensor care processing system and/or device can be used to receive, process, store, and aggregate signals from one or more groups of sensors. The signal or signals from a single sensor or group of sensors can be used to determine that a change of PUM's current operating pattern state has occurred. This pattern change can then trigger the activation of additional sensors and/or the change in the configuration of one or more existing sensors, to update the current monitoring focus to the new PUM state. This trigger can occur in one or more sensors or other components of the sensor care processing system, individually and/or in combination.

For example, if the PUM has been sitting or lying down for some time and then gets up and starts walking, the signal from an active accelerometer in a wearable device, such as a PERS or a smart watch, can detect a change in the PUM's movement, which can trigger the activation of a barometric altimeter sensor, to accurately detect possible falls. The change of PUM's state pattern can also trigger changes in the configuration of the same sensor or sensors that detected such change. For example, the same accelerometer that detected the PUM's change in movement pattern can be automatically re-configured to increase its accuracy and/or its signal update frequency, to detect possible falls and not just movement in general. For example, this can include the monitoring of the gait of the PUM as they move, to in whole or in part, evaluate the potential for that PUM to fall or have another health or wellness event. In some embodiments the change in monitoring focus can be decided, in whole or in part with the invocation of one or more machine learning techniques.

The configuration of the one or more sensors so as to increase the fidelity and/or granularity of their sensing capabilities can enable event sequence detection that can result in more accurate and/or earlier detection of adverse and/or other care related circumstances.

This approach can also be used with one or more redundant sensors to detect sensor failures and avoid resulting false positives or false negatives. The use of multi-dimensional feature sets supports the identification of sensor aberrations or failures, in that the relationship between the feature sets diverges sufficiently so as to create an event representing such divergence. For example, if a sensor has a fly, spider or other insect covering the sensor, this may result in a significant divergence of the data set forming the dimension and the thus the relationship with other collocated sensors.

In some embodiment, when an event is determined to have occurred, one or more processing systems may be invoked in real time to evaluate the relationships of sets of dimensions represented in a multi-dimensional feature set. This can include the use of differing processing systems employing one or more algorithms to differing data sets, dimensions and/or dimension relationships in support of monitoring focus or evaluation of one or more event types and/or patterns, pattern elements and/or behaviors of a person and/or environment under monitoring

In some embodiments, data may be shared across multiple sensors on a contextual instance basis, for example where the data contribution of a sensor is mediated by the care processing in a dynamic manner. This can include passing of data sets from one sensor to another to improve performance of sensor and/or clean that data set and/or act as a feedback mechanism to reduce noise in the data set and the like.

Pattern Processing

Pattern processing can be undertaken with the configuration and/or data sets a set of sensor devices embedded in an environment. The distribution of the processing capabilities and functionalities can include, for example, within the sensor and/or device in which such sensor is embedded, a hub or other device located in the environment, for example a care hub, a network connected to and/or accessible to the sensor, providing access to cloud and/or other accessible processing capabilities, including specialized processing systems in any arrangement.

Many sensors incorporate feature extraction techniques that identify specific characteristics of the data, and in some embodiments communicate only these features. Such sensors may be incorporated into the care processing, however these features may be then be validated and/or verified by other sensors in the same environment to minimize false positives. Some sensors can be configured with additional feature sets, such as those features generated with digital twins using, for example predictive techniques and/or machine learning capabilities.

In some embodiments, the system monitoring and/or care processing can be self-learning, in that initially the environment is sensed to establish a baseline, which may be represented as a pattern framework. Subsequently a set of patterns, for example those included in an HCP representing the PUM care monitoring can be loaded into a care processing system. This can include each pattern having one or more event detection criteria, for example expressed as a multi-dimensional feature set, where the sensing systems can monitor each of these criteria both independently and in aggregate. In some embodiments, probabilistic methods are employed, such that an independent event detected will cause care processing to predict the probability of other sensors generating corresponding event criteria matching outcomes, such that there may be a consensus algorithm used to determine whether the event sequence is sufficient to instigate further more granulated monitoring, for example increasing the monitoring focus, and/or to cause a trigger or alert to be issues for further escalation.

Care signal processing configuration may be based on and/or derived from a ML model developed form the at least one digital twin representing the environment, PUM and/or stakeholders in any arrangement.

The selection and configuration of an edge device is responsive to the initial deployment of a pattern whereby the edge device can be located in the part of the environment a PUM is occupying and/or is selected based on the sensors in that device and/or is carried by the PUM and/or by other criteria specified in the pattern. In many situations an edge device may comprise a set, for example one device and/or sensor in each part of an environment. For example, in each room in a multiroom environment. In this example, as the PUM moves about an environment the edge device for the pattern operating at the time is selected based on the location changes such that at least one edge device is actively monitoring for at least one event, event sequence or other data that matches the operating pattern. This situation is mirrored in the at least one digital twin operating the same pattern as well as other instances of the digital twin that may be operating other patterns that are deemed likely to be invoked in the near-term time frame.

Some embodiments may have edge devices that incorporate configuration, processing and storage sufficient to retain sensor data that is pertinent to the operating pattern, such that only the data that matches an operating pattern specification is retained and/or stored in an appropriate repository. In some embodiments such data may be used, in whole or in part, for the generation of tokens.

Pattern specifications can include pattern elements, considered as pattern elements, which can be combined to form new and/or derivative patterns. These can include preformatted event and event sequence elements, such that for example, if a data set from a set of sensors, exceeds a threshold for a specific time and, for example, a second set of sensors provides a data set confirming this occurrence, then the pattern matching algorithm will be invoked.

In some embodiments, patterns and/or pattern elements can be mapped to devices, such that the device evaluates the data set and communicates the outcome of such evaluation, for example as a token to a care processing module. Such evaluations and communications can be undertaken even though the device may not be currently acting as an “edge” sensor in an array of sensors controlled by a care processing system. In this example, the care processing may dynamically integrate the communications from this sensor and change the status of this or other sensors, as well as configuring further sensors to verify, validate and/or provide additional sensing data that conforms to the operating pattern. This can include definitions of typical patterns for a specified HCP, pattern, pattern element, where at least one device is configured on a dynamic basis to provide at least one “edge” of signal for an event.

In some embodiments, at least one edge device can trigger other sensors, devices and/or systems for verification or other data sourcing.

In some embodiments, such care signal processing across multiple devices for “edges”, which may comprise multiple data sets from multiple devices across a limited time span, for example as a multi-dimensional feature set, can provide enhanced accuracy as to event sequence identification and decrease likelihood of false positives.

This approach can distribute computational load across multiple local and remote processing resources so as to improve efficiency for processing a large number of signals in a complex system.

Selection of data from at least one sensor can be triggered by at least one algorithm such that each data set is then written to a distributed ledger in a sequence representing an event, providing verification of the occurrence of the event. In some embodiments, the identity of the actual event may be obscured.

In some embodiments, there can be integration of active scan sensors and/or transmitters with passive and/or receiver sensors to create an integrated source data set for the application of one or more algorithm for the detection and identification of care related patterns and/or pattern elements for one or more person.

Care Processing Classifier

One of the many challenges of accurate, timely and responsive care processing and/or evaluation is the classification and typing of the signals being processed into suitable data structures that can inform, instruct, configure, arrange and/or command other system elements. In many current situations care processing is tied to rigid and static rule-based systems where if signal A is received by a system, then rule set B is activated. This rigid approach is often less useful to an actual unfolding situation, where the rule-based response may be inappropriate and constraining to the current circumstances. In many cases these rule sets invoke responses with either too much or too little resource and/or action depending on circumstances.

The approach described herein, includes the use of sets of classifiers for differing signal types and supporting the configuration of sensors in relation to those classified care processing data sets. This can include classifiers for multi-dimensional feature sets, where for example, the classifier may have a classification schema which matches a predominant set of data represented by the multi-dimensional feature set. In some embodiments, digital twins and/or the use of Machine learning techniques may be employed to determine the classification of such data sets, including multi-dimensional feature sets, which may comprise incomplete and/or partial feature sets and/or dimensions and/or dimension relationships thereof. Further many of the common care related circumstances and situations, including those represented as patterns and/or pattern elements, can be anticipated and specified as part of pattern frameworks for the care processing, such that each sensor may contribute to one or more pattern frameworks, that can be used to accurately identify a situation and optimize one or more responses.

In some embodiments, the classifier may be dynamic, in that the classification operations, although generally undertaken in advance of their use, can be responsive to pattern frameworks, and/or pattern elements and/or sensor data, including multi-dimensional feature sets that populate these frameworks to form patterns. For example, a sets of sensor data may be determined by a care processing systems to be part of a pattern, such as motion detected in a bedroom during sleep, followed by use of a bathroom, followed by a return to breathing associated with sleep. This data set may also be considered as part of a further pattern, where the occurrence of this event sequence is related to, for example, changes in the temperature of the environment, external factors such as noise, breathing anomalies, such as sleep apnea and the like.

In some embodiments, patterns may be stored, for example, in a graph database, where for example further predicted pattern candidates may also be stored, such as those from predictive systems and/or digital twins.

For example, an environment which is an enclosed space may be modelled as a Hilbert space or similar, using inner products (x,y) of a set of vectors. In this way a multidimensional model of an environment, including at least one person, may be created and adapted in a dynamic manner in response to the contextual changes in the environment. This may be achieved without the need to monitor in real time all the inputs from multiple sensor arrangements through the sparse sampling of a set of such sensors and the use of at least one sensor as the edge sensor for that environment.

This approach may also be used in the evaluation of multi-dimensional feature sets, where dimensions and dimension relationships may be evaluated to determine actual or potential transitions from one pattern or pattern element to another.

The sampling used by the system may be based on a pattern, pattern element and/or pattern framework, where the HCP of the person being monitored is used, in whole or in part, to determine which of the sensors provide information to the system. This can include specification of the data types, as some devices may include multiple sensors, the frequency and duration of such data, the granularity of the data and the like.

Edge sensors may be dynamically configured and may have such configurations deployed in response to patterns that are stored and managed by the system and/or event sequences that occur in the environment.

In a sparse sampling situation, a random model, within a specific distribution may be employed where the overall environment is in a quiescent state. This may follow a pattern specification, in the form of a pattern, pattern element and/or pattern framework, such that the sparse sampling is configured for differing rates and data sets at specific times and/or for specific durations. These samplings may be responsive to events detected by at least one sensor, where the rate, types and data exchange of the sampling may vary according to those events. For example, if a person is asleep at night with a sparse pattern in operation and an acoustic monitoring device detects that the person is experiencing sleep apnea, the patterns being employed in monitoring may be varied in response.

Health Care Patterns (HCP)

Health care patterns are overarching context for a PUM and generally incorporate the initial care condition of the PUM for which the care system was invoked. A specific HCP is a subset of the overall health journey of a PUM, in that once a condition has reached a stage where monitoring is required, the health condition of the PUM is likely to follow a series of conditions that eventually lead to their recovery from the condition or to home care, hospice, hospitalization, palliative or a terminal care situation. The period of time that a person with a condition under monitoring remains in any specific HCP will depend on may factors, including their own health condition, the support provided to them, the health care available and the like. However, once a condition, such Alzheimer's or similar has been determined to be of such concern that care monitoring is required, unless or until methods are found to reverse the situation, there is an inevitable decline in the health of the PUM. The HCP is a quantized specification of the states of change and/or decline that such a PUM may undergo. In some embodiments these states are represented by one or more operating patterns as illustrated in FIGS. 2 and 3.

In some embodiments, there is a HCP in operation, which can include sets of pattern elements and/or patterns representing the key indicators in the form of data sets that can be monitored for a PUM in environment. For example, in the case of a PUM with emphysema, acoustic monitoring of their breathing patterns may be essential.

The HCP can be managed by the system and provide the overall framework for the monitoring and care, with each of the stakeholders, including the PUM, friends and family and the health care professions involved in the diagnosis, monitoring and care of the condition to be monitored involved.

In some embodiments an HCP can include one or more patterns which may be considered as stable “plateau” of the health care journey of the PUM, where the elapsed time that a PUM is in such a condition may vary from person to person. The HCP and patterns and pattern elements included within it can include those events and event sequences that are behavioral indicators that the state of the monitored condition. This can include monitoring for change in the specified condition and/or identification of new conditions. Such changes may be gradual or abrupt, and as such the degree of advance notice may vary.

One or more HCP may represent the journey of a PUM from an initial diagnosis of a condition that requires monitoring, though the stages of their health journey to their recovery or ultimate, eventual terminal decline.

### Transitional Behaviors

A PUM may exhibit one or more behaviors are indicators that the pattern currently operating, representing the behaviors of the PUM, is about to change. For example, if there is an increase in coughing, change in breathing patterns, increased use of spray or breathing assist, this can indicate that the PUM is having an increasing difficulty, and as such is transitioning from one pattern, for example “stable breathing pattern” to another, for example “Breathing trouble”, which for example may form part of an HCP for a PUM who is being monitored for emphysema. These behaviors may be represented as feature sets comprising the sensor and/or device data that may be designated as transition feature sets which are indicators of the change from one pattern to another.

The detection of these behavior changes may be direct, for example through use of FMCW or other active or passive sensors detecting PUM breathing patterns, which for example could be configured as edge sensors and may validated or verified by other sensors such as acoustic sensors, for example MEMS microphones, that detect the variations in the breathing patterns.

In some embodiments, digital twins may be used as part of the transition detection where the current operating pattern is deployed and the data from the sensors is incorporated. These data sets may then be compared with the data sets from the anticipated patterns that form the HCP, for example if “stable breathing pattern” is operating and the anticipated pattern is “breathing trouble”, then one or more matching and/or comparison algorithms may be employed to evaluate the likelihood that this transition is occurring. A digital twin, or set thereof, may compare multiple potential patterns so as to assess the most likely transition. Such evaluation can include using one or more machine learning techniques to identify likely trends and potential transitions.

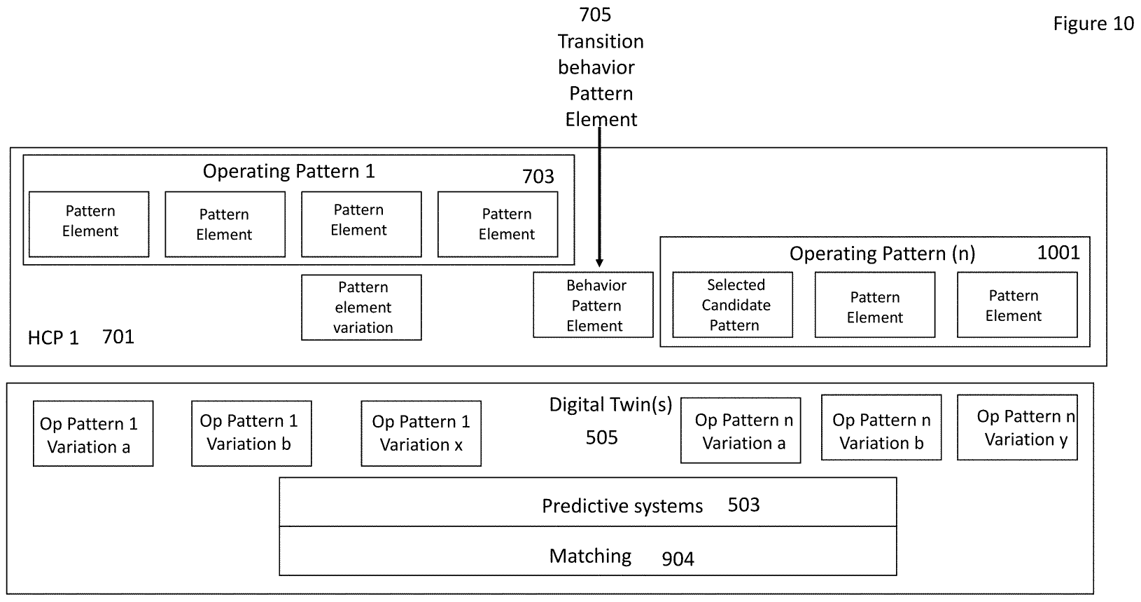

FIG. 10 illustrates one or more digital twins (505), operating in cooperation with the HCP (701), comprising operating patterns (703) and potential operating patterns (1001) where the digital twins comprise one or more operational pattern variations which represent potential variations, based on differing simulated and/or projected sensor data, that can then be matched, using for example, predictive systems (503) and/or matching systems (904), to ascertain based on, at least in part the behavior transition pattern element (705), the most likely and suitable operating pattern (1001), which represents most accurately the care and well-being state of the PUM. The digital twin may then continue such variation projection and/or prediction as the care journey of the PUM unfolds.

In some embodiments, the transition may from one operating pattern to another may result in an alert or event being generated and communicated to an appropriate set of stakeholders, for example a doctor, pharmacy, carer, relative and the like. If the pattern is known as part of an HCP, where the transition is part of a health and wellness voyage that is well understood, then the operating pattern may change and the sensors, devices and/or system configured for that pattern. In the case where the transition represents an immediate and/or potential significant risk of the wellness and health of the PUM, the event and/or alert may be such that emergency and/or other stakeholder are notified. For example, if the breathing of the PUM is not detected indicating a potentially life threating situation.

In some embodiments the relationship between the dimensions of a multi-dimensional feature set can provide indications of the changes in behavior of a PUM through the evaluation of these relationships. For example, if dimension A, representing data from one or more sensor that is detecting breathing of the PUM and dimension B representing data from one or more sensor detecting coughing through, for example acoustic sensors, such as MEMS microphones has a relationship of N where N represents, for example the number of coughs per breath over a time period and that relationship increases, then this may be an indicator of a behavioral change. In this example another dimension may involve the position of the PUM's body in relation to a vertical or horizontal axis. For example, whether the PUM is lying down more than they are sitting or standing the relationship of this dimension to the other dimensions.

In some embodiments, dimensions may comprise differing combinations of sensor data. For example, a dimension resenting a behavior such as coughing may include breathing monitoring sensor data, wearable device sensor data and/or acoustic sensor data. Each of these data sets may have integrated weightings or rankings that impact the overall value of the dimension.

In some embodiments, the detection and/or identification of transition behavior patterns can incorporate one or more machine learning techniques, including regression learning, neural networks and the like, whereby the data set representing one or more behaviors including the one or more feature sets that sensors and/or devices are configured to recognize, can represent transitions between a pattern or pattern element and another pattern and/or pattern element. This can include multiple such patterns and/or pattern elements with differing ranking s based, at least in part, on the relative probability and/or likelihood based on similar circumstances that may be occurring.

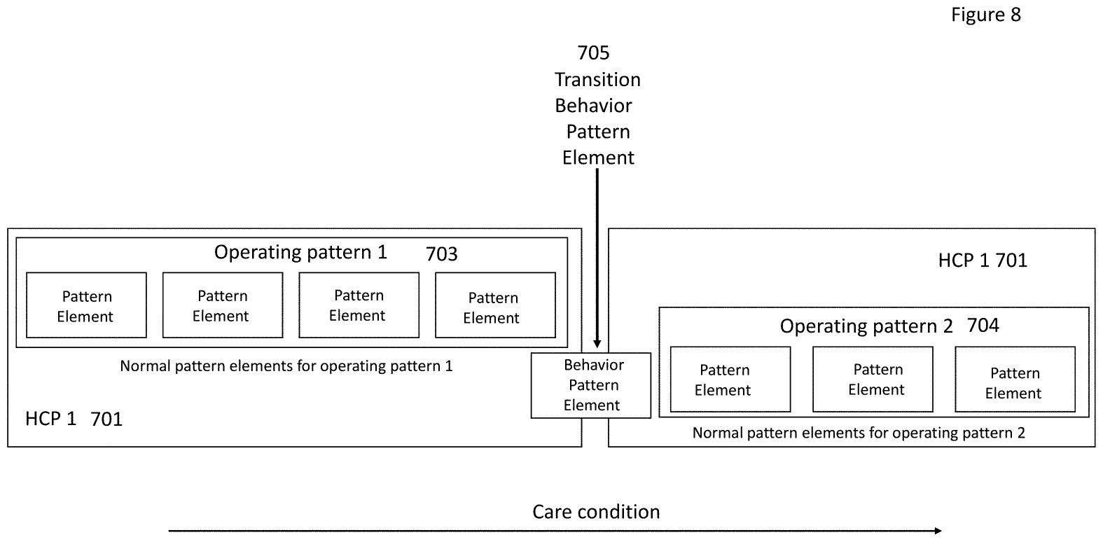

FIG. 7 illustrates a transition state between two operating patterns (703 and 704 respectively), each comprising a set of pattern elements, within an HPC (701), where a behavior pattern represents the transition (705) between the two operating patterns within the HCP.

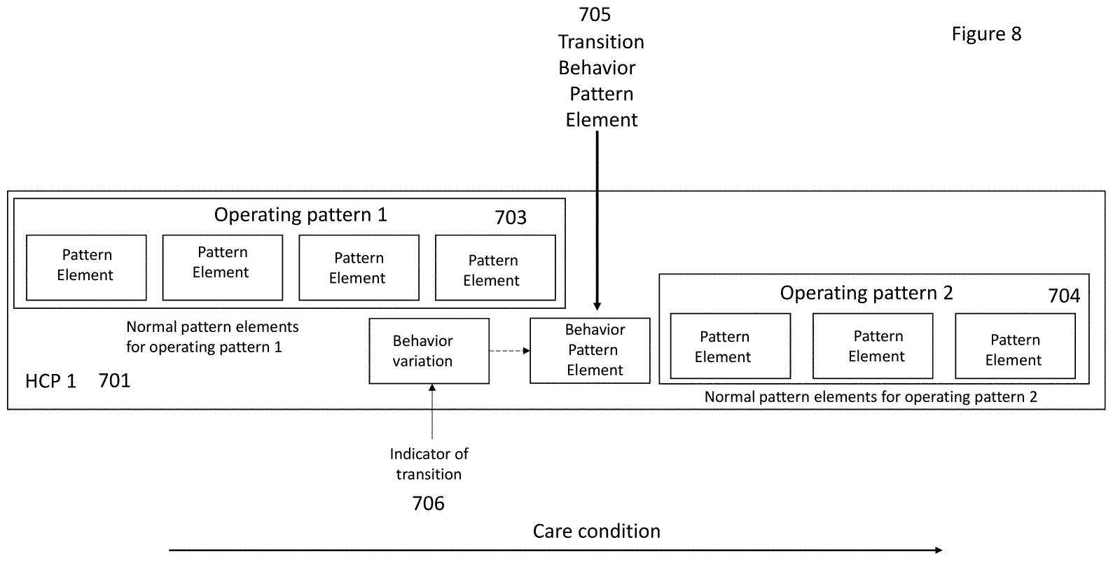

In FIG. 8, a behavior pattern variation is identified (706), and represents a precursor to the transition behavior pattern (705), providing, in whole or in part, an advance notice of that forthcoming change in PUM (105) care condition. This data can be identified through the monitoring of a single PUM (105) and/or can be identified through monitoring of multiple PUM with the same or similar HCP. For example, this can be done through the use of digital twins and/or ML/AI techniques.

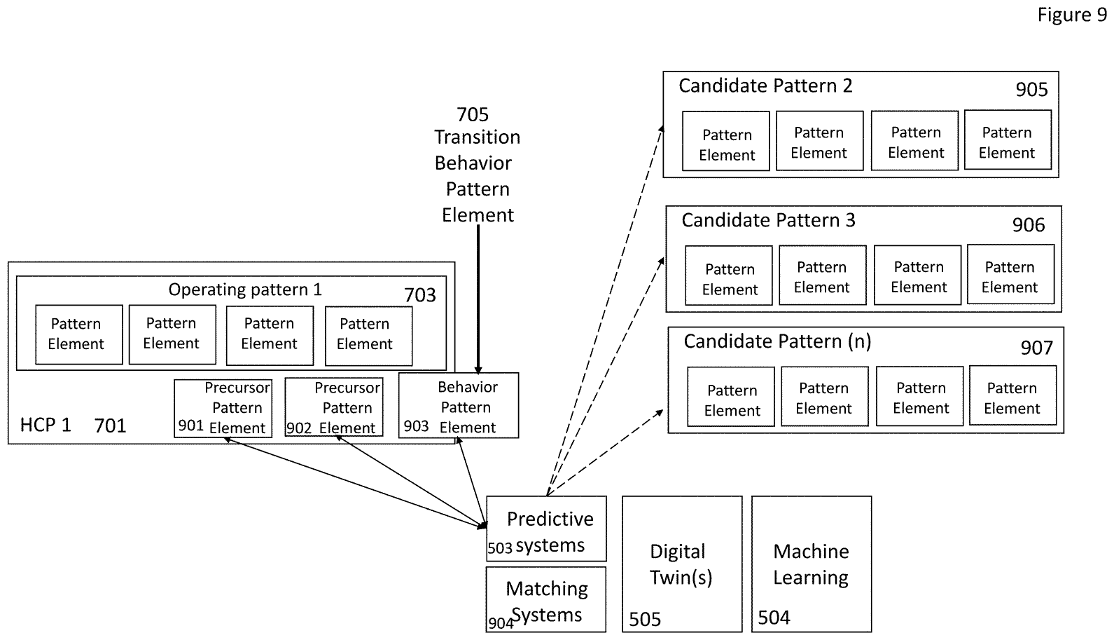

FIG. 9 Illustrates the use of predictive (503) and matching (904) systems, which when embodied can, for example, include one or more digital twins (505) and machine learning modules (504), where a series of candidate patterns (905, 906, 907) are evaluated by the matching systems (904) as the most likely to match the transition behavior pattern (705). In this example, there are two pattern elements that represent precursors (901,902) to the behavioral change represented by the transition behavior pattern element (705), where for example these pattern elements include behavioral attributes that the PUM is exhibiting, that although common to the operating pattern and the pattern elements thereof, can be more accentuated or have other variations that are indicative of change, In some embodiments monitoring focus may be varied to further identify and/or validate such behavior change. In some embodiments, one or more of the candidates (907) may be part of a differing HCP. In some embodiments, these HCP may have a degree of correlation, for example all are associated with a PUM having breathing problems and/or each of the HCP may have differing care focus.

As illustrated in FIG. 9 this can include the use of digital twins (505) in combination with predictive (503) and matching (904) modules to evaluate pattern variations, such as those of precursors (901,902) so as to identify and/or validate a transition behavior pattern element (903), and the transition to One or more candidate operating patterns.

Pattern Frameworks

A pattern framework is a specification that is based in part on the behavior patterns, which can be represented by pattern elements, of a person in an environment. This framework is coupled with the HCP for that person, such that a series of potentially overlapping behavior patterns that typically represent a person's traversing a HCP can be represented in such a framework. For example, if a PUM has for example emphysema, the set of pattern frameworks will include the typical behaviors and timeframes for that condition, the mitigation of the condition based on the various medicines, treatments or other assistance provided, the typical behavioral aspects of the PUM with such a condition, events, event sequences, triggers and other data sets indicating a forthcoming or actual change in their circumstances and the like.

The framework can include those predictive indicators, expressed as event sequences and/or pattern elements, that represent a person changing from one behavior pattern to another, for example a decline or increase in their health condition that is being monitored under care. In some embodiments, this may be represented by a multi-dimensional feature set that comprises one or more dimensions.

A pattern framework may include and/or in part be created by a set of pattern elements, which can be defined as those sensor data, including multi-dimensional feature sets, that form a set of events, generally in a sequence. These sensor data sets can indicate the various changes in state of the sensors and the environment which they are monitoring. However, the behavioral aspects of the PUM are an essential part of the pattern framework, in that these specifications describe the activities of the PUM, providing the context for the sensor data sets, and consequently providing the effective monitoring of the person under care.

One advantage of this approach is the use of the behavioral specifications, for example represented as pattern elements, within a pattern by the care processing system, to arrange and configure sensor sets to focus on a PUM and their current activities, including to verify the specific activity and to identify behavior sets that are indicative of changes in the care state of that PUM. This ability to identify the likely precursors to a care event that requires or demands intervention is essential to the well-being of a PUM. This approach removes the reliance on the PUM self-identifying a potentially significant care event and incorporates the necessary event and alert management systems to communicate to other stakeholders involved in the care of a PUM awareness of a situation.

The pattern framework is initially instantiated, at least in part, on the care condition that has been diagnosed and which forms the initial specifications of the pattern framework as part of the HCP. The set of potential conditions that can be monitored is extensive, however the majority of these are related to the age of the PUM, and as such can be grouped into age specific HCP. These groupings may also be based on the type of care monitoring, for example breathing related, memory impairment related, degenerative disease related and/or the like. In such frameworks, the pattern elements that can comprise such frameworks can be sensor data centric and/or PUM behavior centric. These aspects may be arranged to as to create a pattern framework that is suitable for the care condition being monitored.

A pattern framework that is initially instantiated will, over the course of time, become further populated with data sets from the sensors conforming to either or both of the sensors' data sets and the behavior data sets. This can evolve the initial pattern framework into an operating, active personal pattern that is specific to and for a PUM and the stakeholders and environment with specified relationships to that PUM.

FIG. 4 illustrates the pattern frameworks (401) that can represent one or more HCP (101), where each pattern framework is populated by one or more pattern elements (102), forming one or more operating pattern(s) (103). An HCP (101) may comprise multiple pattern frameworks (401) and/or each pattern framework may comprise multiple pattern elements (102) that form one or more operating patterns (103) in any arrangement.

A further aspect is the instantiation of a digital twin incorporating the initial pattern framework. This digital twin and multiple instantiations thereof, may then be populated with the data sets from the sensors, at any level of granularity, and can be used in conjunction with machine learning techniques to predict behaviors and initiate with the care processing systems, new patterns, arrangements and/or configurations of sensors and transitions to differing operating patterns and/or HCP in any arrangement.

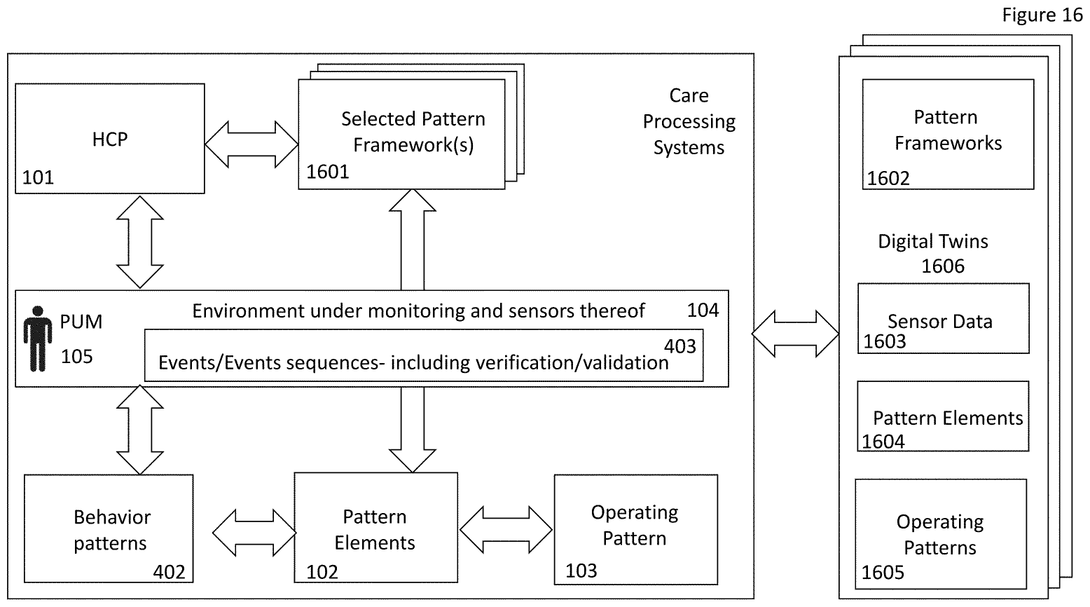

FIG. 16 illustrates one or more digital twins (1606) comprising pattern frameworks (1602), sensor data (1603), pattern elements (1604) and operating patterns (1605) which can represent potential states of a PUM (105) and the environment in which they are monitored (104).

This approach provides for the contextualization of sensor data sets that represent behavioral characteristics and the metrics thereof which is essential to effective, efficient and responsive care management.

Typing of Patterns

In some embodiments an HCP may have a number of patterns that can be deployed which represent the likely behaviors of a PUM in an environment. This can include patterns that are predicted and/or are same or similar to those of other HCP that have common monitoring specifications. For example, if a PUM has condition A and the HCP for that condition comprises patterns A,B,C,D etc., and the PUM under monitoring has a high correlation with those patterns, then such an HCP may be used for another PUM with the same condition. In this example the Patterns A,B,C,D etc. are represented as pattern frameworks comprising pattern elements that represent PUM behaviors without the sensor data sets. In this manner these pattern elements may be populated by the PUM sensor data sets as they traverse the pattern elements and patterns of that HCP. The set of patterns representing the behaviors of a PUM in an environment can be applied across multiple HCP. There may be considerable overlap of patterns, where for example a PUM has multiple care conditions, although one may predominate and as such is the focal point of the monitoring.

Some behavior patterns may be classified in terms of the behavioral routine that a PUM undertakes, for example sleep, exercise, visit to or by a stakeholder, travel, medicine ingestion, therapy, a procedure and the like. Such a classification schema can be arranged as, for example, an ontology, taxonomy, hierarchical or any other arrangement. In some embodiments, the pattern execution and the PUM having undertaken such a pattern and/or set of patterns may be recorded in a suitable repository and/or appropriate distributed ledger. This may typically be the case where medications or specific behaviors highly related to the well-being of the PUM are concerned. Such recordation's may include the tokenization of these patterns.

In some embodiments this may include monitoring of compliance with a treatment plan, including the regular taking of prescribed medicines or other pharmaceutical compounds and/or regular execution of prescribed activities such as therapy-related physical exercise, sleep patterns, eating patterns and the like. This may include other compliance, such as those of an insurance provider, whereby the insurance coverage is, in part, determined by the behavioral compliance of the PUM and/or stakeholders and environment of that PUM. Further compliance may be determined by contracts and/or other specifications that are part of the overall care monitoring arrangements, some of which may be legally binding, and/or may translate into commercial and/or business obligations. In some embodiments this can include court ordered behaviors and activity regimes. In some embodiments such obligations and compliance may form, in whole or in part, a smart contract which is recorded in one or more distributed ledger.

The patterns and/or pattern elements employed comprise specification sets for behaviors that represent the sets of events and sensor data that represent those behaviors. These specifications can encompass multiple sensors, environments and/or stakeholders. The specifications may be dynamically varied in response to changes in circumstances of the environment and the PUM. This can include increasing or decreasing the fidelity of the sensors through variable configurations, for example using monitoring focus module. In some situations, this may involve substitution of one pattern for another.

For example, there may be a pattern operating in an environment which may include configuration of a set of sensors, which can be a subset of all the available sensors in that environment. This pattern may have further patterns that are prearranged, such as in an elastic repository, which can be local and/or remote to the sensors and/or environment, such that if the care processing detects a behavior that matches certain criteria, for example excessive breathing, heightened heart rate, acceleration in one or more axis and the like, the current operating pattern, may in whole or in part, be replaced by a cached pattern in a manner that is contiguous. This can include the activation, configuration and/or reception of data from the sensors operating under the previous pattern and/or may include the activation, configuration and reception of data from additional sensors, for example acoustic, video, radar, carried, worn and/or ingested sensors. In the same manner the number and types of sensors may be increased or decreased as determine by the operating pattern. In some embodiments such changes in patterns and/or pattern elements may be initiated by a transition behavior pattern, which is represented by variations in the one or more dimensions of one or more multi-dimensional feature sets.

In some embodiments, a pattern or pattern elements can include specifications that assign priorities to one or more sensors, change state and/or configuration of sensor, for example to conserve battery capacity and the like. For example, GPS may be put into a sleep state when location is known, for example home, and may be activated when an exit trigger is detected.

In some embodiments, care processing systems may invoke different patterns using the same and/or segmented sets of sensors for monitoring. These patterns may be operated by one or more digital twins, where the data from the sensors may comprise, historical, estimated, predicted and/or actual real time or near real time data sets in any arrangement.

Patterns and/or pattern elements may be categorized and typed according to one more ontologies, taxonomies or other organizing principles. The care processing system may create new patterns and/or pattern elements based on existing patterns, for example using machine learning techniques.

In some cases, there may be what appear to be data sets that can apply to a multitude of patterns, such as a movement detection, however given the nature of the care processing systems, such data sets representing an event, although potentially monitored, can be considered in the context of the other events and data sets representing them that occur and/or are likely to occur with that initial event.

### Example Embodiment

A mobile Personal Emergency Response System (PERS) device is intended for elderly persons or for persons with physical disabilities to request help or emergency services by pushing an emergency button in the PERS device. These devices typically include an emergency button, a speaker, a microphone, and wireless communication capabilities, including limited wireless phone functions which are used to connect the person with emergency personnel or a caretaker using voice. In some cases, a PERS device also contains sensors and software that uses the sensors' signals to detect events such as falls, and to automatically trigger an emergency call and/or report the event to a central server when such events occur. They may also include location detection sensors, such as GPS, radio frequency triangulation, beacon readers or others, which allow the PERS device, or the system that it connect with, to trigger emergency or other events, for example, when the person leaves a pre-determined area (Geo-fence), when they stop moving for a long enough period of time or under other location and/or movement related circumstances.

One problem with PERS devices configured with these and other sensors is that keeping all the sensors active and processing their signals most of the time, to effectively detect relevant events, can drain the devices' battery quickly, which reduces their practicality in real-life situations. This problem can be solved and the performance and accuracy of the PERS device's functions can be improved by applying the concepts described here. For example, under normal circumstances (the quiescent state), most sensors in the device can be configured to remain dormant, except for the accelerometer and the software within the PERS device can be listening only for signals from the accelerometer that indicate movement above a pre-defined threshold, indicating that the person changed their status from passive to active. At this point other sensors, such as the altimeter and the microphone in the device can be activated, the configuration of the accelerometer and the threshold for its signals can change, and the software can switch to a different set of detection logic, creating a different configuration, appropriate for detecting the most likely events under the person's new state. Additionally, the location detection sensors and the geo-fence logic can be activated. A geo-fence can also trigger a new change of configuration, for example, when a “going outside” situation is detected, the operation parameters and the event detection logic for the accelerometer and altimeter can be changed, in order to detect falls under the dynamics of walking outside or can be deactivated, if a “moving in a car” situation is detected, based, for example, on the combination of location changes and accelerometer signals. With this approach, sensors, processing, and communications functions are only used when a detected pattern indicates that they are required, resulting in reduced power consumption. Additionally, dynamically changing sensor configurations and detection logic allows for increased event detection accuracy.

An implication of using a PERS device as the single way of detecting risk-related events such as falls is the limited precision that results from a co-located set of sensors in a reduced size portable device. This makes it difficult to avoid false positive and false negative event situations. This can be improved by combining the PERS device's combinations of sensor configurations, data processing and detection logic with those of devices located within the same environment, but outside of the PERS device.

For example, the PERS user's home may be equipped with additional sensors, such as cameras, smoke detectors microphones and the like. The signals from these sensors can be combined with the PERS device's sensors' signals, as well as other devices, such as voice recognition-enabled speakers (smart speakers), as way to make a PERS system more effective. This combination of PERS device data and other sensor data can provide a more accurate, complete and actionable data set as to the state of the PUM. For example, there may be specialized and/or general devices that can interoperate with a PERS device either directly and/or through a specialized device, such as a care hub. This can include one o more communications with such other devices within the user's home, connected to the PERS device using a wireless communications mechanism such as Wi-Fi or Bluetooth, and may include one or more local and/or remote server. This combination can be used to determine more precisely the user's state, based on known user behavioral patterns, such as typical locations and activities within the home, and the signal patterns that those activities produce in the sensor arrangement of the home sensors and the PERS device's sensors.

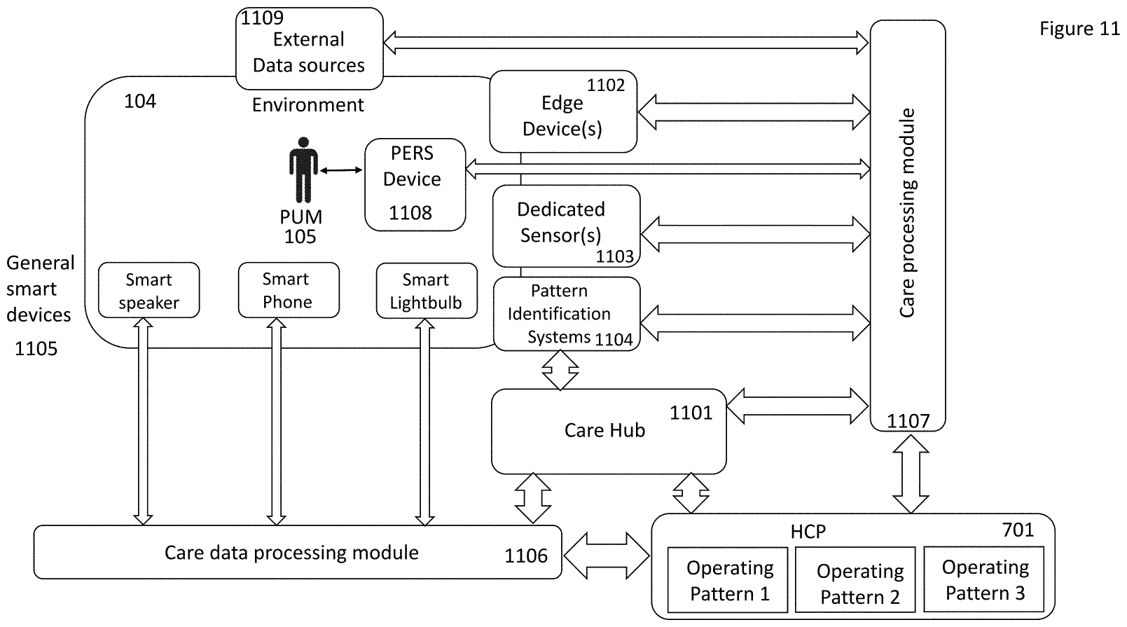

For example, as illustrated in FIG. 11, a PERS device (1108) being worn by a PUM (105) in an environment (104) generates data that is combined with further data generated by general smart devices co-located in the environment (1105), dedicated sensors (1103), external data sources (1109), such as weather, traffic, emergency situations and the like, and/or other sensors, including those designated as edge devices (1102). These data sets may be processed by care data processing module (1106) and/or care processing modules (1107), which in conjunction with pattern identification systems (1104) can form multi-dimensional feature sets, which, can represent in whole or in part, pattern elements and/or operating patterns of the HCP (701). A care hub (1101) may operate to support such aggregations, integrations, processing and communications.

Some of the devices and/or the servers in this kind of configuration may include machine learning or statistical mechanisms as adaptive methods to identify patterns that indicate changes in the state of the user or the environment and to select sensor configurations more accurately and/or trigger events or alarms. Signals transmitted by the sensors in the PERS devices and the user's home can be stored and used in the server for training machine learning systems and/or to feed digital twins of the user and the environment, allowing for adaptations to changes in the user's behaviors and in the environment, and for more accurately predict possible outcomes and prepare emergency and support resources for them.

In some embodiments, a device and incorporated sensors may be able to ascertain one or more care related biometric information sets, which in isolation provide some information for monitoring, however in combination with the HCP and other pattern management incorporated into the system may become informing as to the overall state of the person under care.

In some embodiments, an edge device may be configured as a hub so as to aggregate one or more data sets from sensors and/or coordinate one or more configurations for such sensors. This can include providing processing for such sensors, subject to the capability of the edge device. In some embodiments a care hub may be designated as an edge device.

The use of distributed processing capabilities across multiple sensors, devices, modules and/or systems can include systems deployed at the monitored environment and/or cloud or other remote capabilities, in any arrangement. In some embodiments, the monitoring system may be configured so as to have multiple levels of redundancy to account for loss of communications, power or other critical capabilities. The configuration, in some embodiments, may employ standard redundancy and resilience techniques to ensure minimal monitoring functions are operational for a sufficient period that enables additional external assistance, such as human intervention to be available to the PUM.

This can include the use of backup power systems, multiple redundant communication systems, physically local assistance, such as neighbors or friends and the like.

In some embodiments, ingestible and/or implantable sensors may be incorporated to provide sensors data sets. These sensors may form part of a set of sensors that are worn by a PUM, such as for example a PERS, smart clothing, smart watch and the like, where these devices can receive the data from the implanted and/or ingested sensors. In some embodiments the PERS may provide a secondary power source to these ingested and/or implanted devices. The PERS or other devices may poll the implanted and/or ingested sensors in a manner that preserves the power sources of these devices using a range of techniques, including for example, RF, near field, inductive charging and the like.

One aspect is the relationship between a device, such as a PERS and the ingested and/or implanted sensors, where the data sets from the individual sensors may be directed to one or more other device, such as a medical monitor, with the PERS or other worn or carried device providing a communications path to the other device, In this manner the sensor may use low power low range communications techniques and the PERS or other worn or carried device may provide a higher power and/or longer range communications capability. In some embodiments the nature of the sensor data may be such that the PERS or other worn or carried device may not have access to the sensor data and may encrypt such data for onward transmission to specifically identified, authorized and/or authenticated other devices. In some embodiments, such sensor data may be communicated to other devices in a manner that protects the Personal Identifying Information (PII) or HIPAA data, such as Protected Health Information (PHI) of the PUM. In some embodiments the PERS may communicate the sensors data sets to a care hub, which may in turn operate to further anonymize the data set from the sensor, for example using TOR or other internet routing technologies, to reduce any potential identification through the location and/or routing of the packets that represent that sensor data and/or the PERS of the PUM.

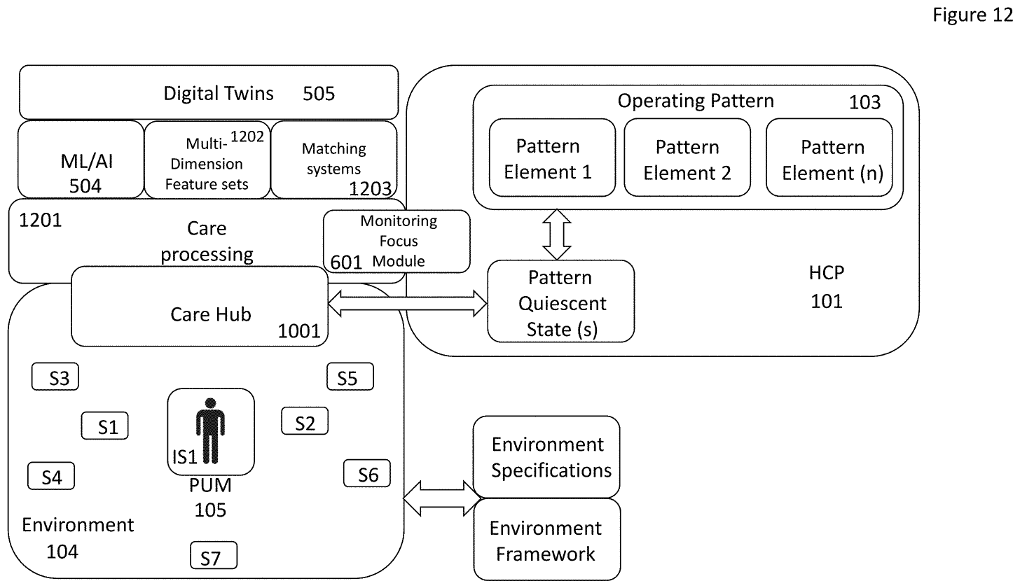

FIG. 12 Illustrates a PUM (105) in an environment (104) that includes sets of sensors Si through S7 and ingested/implanted sensor (IS1), where in this example a care hub (1001) provides communications capabilities to the sensors and provides care processing capabilities (1201), which may be local and/or remote to the care hub. Care hub and care processing integrate and communicate with digital twins (505), machine learning (504) and/or matching systems (1203) including multi-dimensional feature sets (1202) in support of PUM wellness and care monitoring. The state of the PUM may be represented by one or more patterns, for example operating patterns (103) including those patterns that represent the quiescent state (1204) of the PUM and environment within the HCP (101). Changes in such states may be identified by the care hub and/or care processing, which may in turn invoke the monitoring focus module (601) to change the configuration of the one or more sensors and/or processing systems so as to more accurately determine the state of the PUM.

The previous description of the embodiments is provided to enable any person skilled in the art to practice the disclosure. Thus, the present disclosure is not intended to be limited to the embodiments shown herein, but is to be accorded the widest scope consistent with the principles and features disclosed herein.

What is claimed is:

1. A system to monitor a person under care by a stakeholder, comprising:
a plurality of environmental sensors configured to monitor the person under care, and to provide a detected data set representing behaviors of the person under care in an environment;
each of the behaviors is represented by a multi-dimensional feature set forming part of a health care profile for the person under care;
a care processing system comprising:
a transceiver configured to receive the detected data set,
a non-transitory computer-readable storage medium configured to store a quiescent data set, the quiescent data set representing previous quiescent behaviors of the person under care in the environment, and
at least one hardware processing unit to determine a wellness or care event for the person under care by comparing the detected data set and the quiescent data set,
when the wellness or care event has occurred, the care processing system is configured to change a state of the plurality of environmental sensors or notify the stakeholder.

2. The system of claim 1, wherein the multi-dimensional feature set includes manifolds, Hilbert spaces or other representation capable of storing the detected data set from the plurality of environmental sensors.

3. The system of claim 2, wherein the detected data set is from a temperature sensor, acoustic sensor or motion detector.

4. The system of claim 2, wherein the detected data set represents an aggregate data from the plurality of sensors.

5. The system of claim 4, wherein the aggregate data is expressed as ratios, functions, algorithms, or spatial expression.

6. The system of claim 2, wherein the detected data set is from a breathing sensor, or heart-rate sensor.

7. The system of claim 6, wherein the quiescent data set represents: sleeping, eating, bathroom use, or exercise.

8. The system of claim 2, wherein the changing the state of the plurality of environmental sensors alters a monitoring focus of the environmental sensors.

9. The system of claim 8, wherein the monitoring focus increases the fidelity or granularity of the environmental sensors.

10. A system to deploy a pattern representing a health state of a person under care by a stakeholder, comprising:
a plurality of environmental sensors configured to monitor the person under care, and to provide a detected data set representing behaviors of the person under care in an environment;
each of the behaviors is represented by a multi-dimensional feature set forming part of a health care profile for the person under care;
a care processing system comprising:
a transceiver configured to receive the detected data set,
at least one hardware processing unit to determine a variation in the detected data set indicating a transition state between a first pattern and a second pattern within the health state representing a wellness and care state of the person under care, and
the care processing system is configured to change a sensor configuration of the plurality of environmental sensors to adjust for the transition state.

11. The system of claim 10, wherein the multi-dimensional feature set includes manifolds, Hilbert spaces or other representation capable of storing the detected data set from the plurality of environmental sensors.

12. The system of claim 11, wherein the detected data set is from a temperature sensor, acoustic sensor or motion detector.

13. The system of claim 11, wherein the detected data set represents an aggregate data from the plurality of sensors.

14. The system of claim 13, wherein the aggregate data is expressed as ratios, functions, algorithms, or spatial expression.

15. The system of claim 11, wherein the detected data set is from a breathing sensor, or heart-rate sensor.

16. The system of claim 15, wherein the quiescent data set represents: sleeping, eating, bathroom use, or exercise.

17. The system of claim 11, wherein the changing the state of the plurality of environmental sensors alters a monitoring focus of the environmental sensors.

18. The system of claim 17, wherein the monitoring focus increases the fidelity or granularity of the environmental sensors.

19. A system to monitor a person under care by a stakeholder comprising:
a plurality of environmental sensors configured to monitor the person under care, and to provide a detected data set representing a care state of the person under care in an environment;
a care processing system comprising:
a transceiver configured to receive the detected data set,
at least one hardware processing unit to identify and determine a care signal that represents the care state of the person under care, the care signal comprising a multi-dimensional feature set; and
the care processing system is configured to respond to the care signal involving the stakeholder.

20. The system of claim 19, wherein the multi-dimensional feature set includes manifolds, Hilbert spaces or other representation capable of storing the detected data set from the plurality of environmental sensors.
